(19) United States
(12) Patent Application Publication
(10) Pub. No.: US 2025/0351722 A1
(43) Pub. Date: Nov. 13, 2025

(54) PHOTOELECTRIC CONVERSION ELEMENT, IMAGING ELEMENT, OPTICAL SENSOR, AND COMPOUND

(71) Applicant: FUJIFILM CORPORATION, Tokyo (JP)

(72) Inventors: Saika Izumi, Kanagawa (JP); Yosuke Yamamoto, Kanagawa (JP); Yuko Suzuki, Kanagawa (JP); Yasunori Yonekuta, Kanagawa (JP)

(21) Appl. No.: 19/279,388

(22) Filed: Jul. 24, 2025

Related U.S. Application Data
(63) Continuation of application No. PCT/JP2024/010978, filed on Mar. 21, 2024.

(30) Foreign Application Priority Data
Mar. 24, 2023 (JP) 2023-048475

(51) Int. Cl. H10K 85/60; C07D 493/04; C07D 495/04; C07D 513/04; H10K 30/30

(52) U.S. Cl. CPC H10K 85/657; C07D 493/04; C07D 495/04; C07D 513/04; H10K 85/615; H10K 85/654; H10K 85/655; H10K 85/6574; H10K 30/353

(57) ABSTRACT

The present invention provides a photoelectric conversion element which exhibits low electric field strength dependence of a response speed in a case of receiving blue and green light. In addition, the present invention provides an imaging element, an optical sensor, and a compound, which are related to the photoelectric conversion element. The photoelectric conversion element of the present invention includes, in the following order, a conductive film, a photoelectric conversion film, and a transparent conductive film, in which the photoelectric conversion film contains a compound represented by Formula (1). In Formula (1), D represents a group represented by any of Formula (2) to Formula (6).

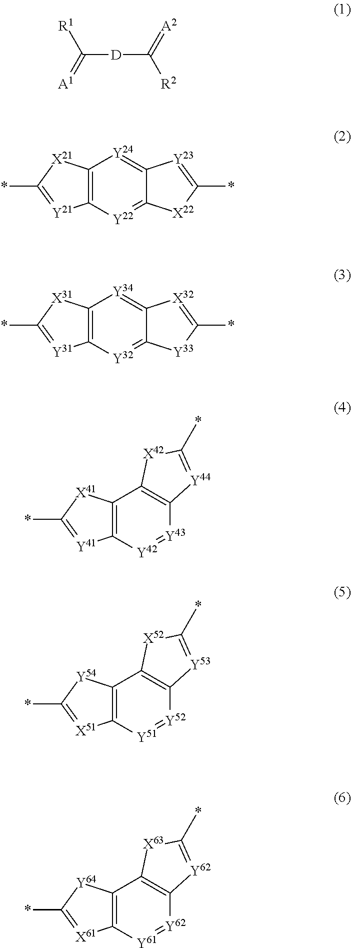

### CROSS-REFERENCE TO RELATED APPLICATIONS

This application is a Continuation of PCT International Application No. PCT/JP2024/010978 filed on Mar. 21, 2024, which claims priority under 35 U.S.C. § 119(a) to Japanese Patent Application No. 2023-048475 filed on Mar. 24, 2023. The above applications are hereby expressly incorporated by reference, in their entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, development of an element (for example, an imaging element) having a photoelectric conversion film has progressed.

For example, WO2014/026224A discloses a photoelectric conversion element containing a specific compound as an electron accepting material.

### SUMMARY OF THE INVENTION

In recent years, further improvements are also required for various characteristics required for a photoelectric conversion element used in an imaging element and an optical sensor, along with demands for improving performance of the imaging element, the optical sensor, and the like.

For example, there is a higher demand for the photoelectric conversion element to have low electric field strength dependence of a response speed in a case of receiving blue and green light (particularly, light having a wavelength of 460 nm). Here, the above-described blue and green light refers to light in a wavelength range of 400 to 600 nm, and “low electric field strength dependence of the response speed” refers to a small change in response speed in a case where a voltage applied to the photoelectric conversion element is changed.

As a result of studying the photoelectric conversion element containing the compound, disclosed in WO2014/026224A, the present inventors have found that there is room for further improvement in electric field strength dependence of a response speed in a case of receiving the above-described blue and green light.

An object of the present invention is to provide a photoelectric conversion element which exhibits low electric field strength dependence of a response speed in a case of receiving blue and green light.

Another object of the present invention is to provide an imaging element, an optical sensor, and a compound.

The present inventors have completed the present invention as a result of intensive studies to solve the above-described problems. That is, the present inventors have found that the above-described objects can be achieved by the following configuration.

[1] A photoelectric conversion element comprising, in the following order:
a conductive film;
a photoelectric conversion film; and
a transparent conductive film,
in which the photoelectric conversion film contains a compound represented by Formula (1) described later.

[2] The photoelectric conversion element according to [1],
in which at least one of Y²¹, . . . , or Y²⁴ in Formula (2) represents —CRs=, at least one of Y³¹, or Y³⁴ in Formula (3) represents —CRs=, at least one of Y⁴¹, . . . , or Y⁴⁴ in Formula (4) represents —CRs=, at least one of Y⁵¹, . . . , or Y⁵⁴ in Formula (5) represents —CRs=, and at least one of Y⁶¹, . . . , or Y⁶⁴ in Formula (6) represents —CRs=, and Rs's each independently represent a group selected from a substituent group S described later,
in a case where a plurality of Rs's are present, Rs's may be the same or different from each other.

[3] The photoelectric conversion element according to [1] or [2],
in which D represents a group represented by any of Formula (2-10) described later, Formula (2-11) described later, Formula (2-12) described later, Formula (3-10) described later, Formula (3-11) described later, Formula (3-12) described later, Formula (4-10) described later, Formula (4-11) described later, Formula (4-12) described later, Formula (5-10) described later, Formula (5-11) described later, Formula (5-12) described later, Formula (6-10) described later, Formula (6-11) described later, or Formula (6-12) described later.

[4] The photoelectric conversion element according to any one of [1] to [3],
in which Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ each independently represent —CR^{Y1}=.

[5] The photoelectric conversion element according to any one of [1] to [4],
in which A¹ and A² are each independently the group represented by Formula (A-1).

[6] The photoelectric conversion element according to [3],
in which Rs represents a group selected from the group consisting of the alkyl group which may have a substituent, the aryl group which may have a substituent, the heteroaryl group which may have a substituent, and the halogen atom, and
in Formula (2-12), Formula (3-12), Formula (4-12), Formula (5-12), and Formula (6-12), two Rs's represent groups different from each other.

[7] The photoelectric conversion element according to [6],
in which Rs represents a group selected from the group consisting of the alkyl group which may have a substituent and the aryl group which may have a substituent, and
in Formula (2-12), Formula (3-12), Formula (4-12), Formula (5-12), and Formula (6-12), two Rs's represent groups different from each other.

[8] The photoelectric conversion element according to any one of [1] to [7],
in which the photoelectric conversion film further contains an n-type organic semiconductor, and
the photoelectric conversion film has a bulk heterojunction structure formed in a state in which the compound represented by Formula (1) and the n-type organic semiconductor are mixed with each other.

[9] The photoelectric conversion element according to [8],
in which the n-type organic semiconductor includes fullerenes selected from the group consisting of a fullerene and derivatives of the fullerene.

[10] The photoelectric conversion element according to any one of [1] to [9],
in which the photoelectric conversion film further contains a p-type organic semiconductor.

[11] The photoelectric conversion element according to any one of [1] to [10], further comprising:
one or more interlayers between the conductive film and the transparent conductive film, in addition to the photoelectric conversion film.

[12] An imaging element comprising:
the photoelectric conversion element according to any one of [1] to [11]

[13] An optical sensor comprising:
the photoelectric conversion element according to any one of [1] to [11]

[14]A compound represented by Formula (1) described later.

[15] The compound according to [14],
in which at least one of Y²¹, . . . , or Y²⁴ in Formula (2) represents —CRs=, at least one of Y³¹, or Y³⁴ in Formula (3) represents —CRs=, at least one of Y⁴¹, . . . , or Y⁴⁴ in Formula (4) represents —CRs=, at least one of Y⁵¹, . . . , or Y⁵⁴ in Formula (5) represents —CRs=, and at least one of Y⁶¹, . . . , or Y⁶⁴ in Formula (6) represents —CRs=, and Rs's each independently represent a group selected from a substituent group S described later.

[16] The compound according to [14] or [15],
in which D represents a group represented by any of Formula (2-10) described later, Formula (2-11) described later, Formula (2-12) described later, Formula (3-10) described later, Formula (3-11) described later, Formula (3-12) described later, Formula (4-10) described later, Formula (4-11) described later, Formula (4-12) described later, Formula (5-10) described later, Formula (5-11) described later, Formula (5-12) described later, Formula (6-10) described later, Formula (6-11) described later, or Formula (6-12) described later.

[17] The compound according to any one of [14] to [16],
in which Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ each independently represent —CR^{Y1}=.

[18] The compound according to any one of [14] to [17],
in which A¹ and A² are each independently the group represented by Formula (A-1).

[19] The compound according to [15],
in which Rs represents a group selected from the group consisting of the alkyl group which may have a substituent, the aryl group which may have a substituent, the heteroaryl group which may have a substituent, and the halogen atom, and
in Formula (2-12), Formula (3-12), Formula (4-12), Formula (5-12), and Formula (6-12), two Rs's represent groups different from each other.

[20] The compound according to [19],
in which Rs represents a group selected from the group consisting of the alkyl group which may have a substituent and the aryl group which may have a substituent, and
in Formula (2-12), Formula (3-12), Formula (4-12), Formula (5-12), and Formula (6-12), two Rs's represent groups different from each other.

According to the present invention, it is possible to provide a photoelectric conversion element which exhibits low electric field strength dependence of a response speed in a case of receiving blue and green light.

In addition, according to the present invention, it is possible to provide an imaging element, an optical sensor, and a compound.

### BRIEF DESCRIPTION OF THE DRAWINGS

FIG. 1 is a schematic cross-sectional view showing an example of a configuration of a photoelectric conversion element.

FIG. 2 is a schematic cross-sectional view showing an example of a configuration of a photoelectric conversion element.

### DESCRIPTION OF THE PREFERRED EMBODIMENTS

Hereinafter, the present invention will be described in detail.

The description of the configuration requirements described below is made on the basis of representative embodiments of the present invention, but it should not be construed that the present invention is limited to those embodiments.

Hereinafter, meaning of each description in the present specification will be explained.

In the present specification, numerical ranges represented by “to” include numerical values before and after “to” as lower limit values and upper limit values.

In the present specification, a hydrogen atom may be a light hydrogen atom (normal hydrogen atom) or a heavy hydrogen atom (for example, a deuterium atom or the like).

In the present specification, regarding a compound which may have a geometric isomer (cis-trans isomer), a general formula or a structural formula representing the compound may be described only in the form of either a cis isomer or a trans isomer for convenience. Even in such a case, unless otherwise specified, the form of the compound is not limited to either the cis isomer or the trans isomer, and the compound may be the cis isomer or the trans isomer.

A bonding direction of a divalent group (for example, —CO—O—) described in the present specification is not limited unless otherwise specified. For example, in a case where Y in a compound represented by a formula “X-Y-Z” is —CO—O—, the compound may be any of “X—O—CO—Z” or “X—CO—O—Z”.

A symbol “*” specified in a chemical formula represents a bonding position unless otherwise specified.

In the present specification, in a case of a plurality of substituents, linking groups, and the like (hereinafter, also referred to as “substituent and the like”) represented by specific reference numeral, or in a case of simultaneously defining a plurality of the substituent and the like, it means that each of the substituent and the like may be the same as or different with each other. This also applies to a case of specifying the number of substituents and the like. In the present specification, the “substituent” includes a group exemplified by a substituent W described later, unless otherwise specified.

### (Substituent W)

The substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heterocyclic group (a heteroaryl group or an aliphatic heterocyclic group), a cyano group, a nitro group, an alkoxy group, an aryloxy group, a silyl group, a silyloxy group, a heterocyclic oxy group, an acyloxy group, a carbamoyloxy group, an alkoxycarbonyloxy group, an aryloxycarbonyloxy group, a primary, secondary, or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a carboxy group, a phosphoric acid group, a sulfonic acid group, a hydroxy group, a thiol group, an acylamino group, a carbamoyl group, a ureido group, and a boronic acid group.

In addition, each of the above-described groups may further have a substituent (for example, one or more groups of each of the above-described groups), as possible. For example, an alkyl group which may have a substituent is also included as the form of the substituent W.

In addition, in a case where the substituent W has a carbon atom, the number of carbon atoms in the substituent W is, for example, 1 to 20.

The number of atoms other than a hydrogen atom in the substituent W is, for example, 1 to 30.

It is also preferable that a specific compound described later does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In the present specification, the aliphatic hydrocarbon group may be linear, branched, or cyclic.

Examples of the above-described aliphatic hydrocarbon group include an alkyl group, an alkenyl group, and an alkynyl group.

In addition, in the present specification, unless otherwise specified, the number of carbon atoms in the alkyl group is preferably 1 to 20, more preferably 1 to 10, and still more preferably 1 to 6.

The alkyl group may be linear, branched, or cyclic.

Examples of the alkyl group include a methyl group, an ethyl group, an n-propyl group, an isopropyl group, an n-butyl group, a t-butyl group, an n-hexyl group, and a cyclopentyl group.

In addition, the alkyl group may be any of a cycloalkyl group, a bicycloalkyl group, or a tricycloalkyl group, and may have a ring structure thereof as a partial structure.

In the alkyl group which may have a substituent, examples of the substituent which may be included in the alkyl group include the groups exemplified as the substituent W. Among these, an aryl group (preferably having 6 to 18 carbon atoms and more preferably having 6 carbon atoms), a heteroaryl group (preferably having 5 to 18 carbon atoms and more preferably having 5 or 6 carbon atoms), or a halogen atom (preferably a fluorine atom or a chlorine atom) is preferable.

In the present specification, unless otherwise specified, an alkyl group moiety in the alkoxy group is preferably the above-described alkyl group. An alkyl group moiety in the alkylthio group is preferably the above-described alkyl group.

In the alkoxy group which may have a substituent, examples of the substituent which may be included in the alkoxy group include the same examples as the substituent in the alkyl group which may have a substituent. In the alkylthio group which may have a substituent, examples of the substituent which may be included in the alkylthio group include the same examples as the substituent in the alkyl group which may have a substituent.

In the present specification, the alkenyl group may be any of linear, branched, or cyclic, unless otherwise specified. The number of carbon atoms in the above-described alkenyl group is preferably 2 to 20. In the alkenyl group which may have a substituent, examples of the substituent which may be included in the alkenyl group include the same examples as the substituent in the alkyl group which may have a substituent.

In the present specification, the alkynyl group may be any of linear, branched, or cyclic, unless otherwise specified. The number of carbon atoms in the above-described alkynyl group is preferably 2 to 20. In the alkynyl group which may have a substituent, examples of the substituent which may be included in the alkynyl group include the same examples as the substituent in the alkyl group which may have a substituent.

In the present specification, an aromatic ring or an aromatic ring constituting the aromatic ring group may be any of a monocyclic ring or a polycyclic ring (for example, 2 to 6 rings or the like), unless otherwise specified. The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) fused aromatic ring structures, as a ring structure.

The number of ring member atoms in the above-described aromatic ring is preferably 4 to 15.

The above-described aromatic ring may be an aromatic hydrocarbon ring or an aromatic heterocyclic ring.

In a case where the above-described aromatic ring is an aromatic heterocyclic ring, the number of heteroatoms included as ring member atoms is, for example, 1 to 10. Examples of the above-described heteroatom include a nitrogen atom, a sulfur atom, an oxygen atom, a selenium atom, a tellurium atom, a phosphorus atom, a silicon atom, and a boron atom.

Examples of the above-described aromatic hydrocarbon ring include a benzene ring, a naphthalene ring, an anthracene ring, and a phenanthrene ring.

Examples of the above-described aromatic heterocyclic ring include a pyridine ring, a pyrimidine ring, a pyridazine ring, a pyrazine ring, a triazine ring (for example, 1,2,3-triazine ring, 1,2,4-triazine ring, 1,3,5-triazine ring, and the like), a tetrazine ring (for example, 1,2,4,5-tetrazine ring and the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzimidazole ring, a benzoxazole ring, a benzothiazole ring, a naphthopyrrole ring, a naphthofuran ring, a naphthothiophene ring, a naphthimidazole ring, a naphthoxazole ring, a pyrroloimidazole ring (for example, a 5H-pyrrolo[1,2-a]imidazole ring and the like), an imidazooxazole ring (for example, an imidazo[2,1-b]oxazole ring and the like), a thienothiazole ring (for example, a thieno[2,3-d]thiazole ring and the like), a benzothiadiazole ring, a benzodithiophene ring (for example, benzo[1,2-b:4,5-b′]dithiophene ring and the like), a thienothiophene ring (for example, thieno[3,2-b]thiophene ring and the like), a thiazolothiazole ring (for example, thiazolo[5,4-d]thiazole ring and the like), a naphthodithiophene ring (for example, a naphtho[2,3-b:6,7-b′]dithiophene ring, a naphtho[2,1-b:6,5-b′]dithiophene ring, a naphtho[1,2-b:5,6-b′]dithiophene ring, a 1,8-dithiadicyclopenta[b,g]naphthalene ring, and the like), a benzothienobenzothiophene ring, a dithieno[3,2-b:2′,3′-d]thiophene ring, and a 3,4,7,8-tetrathiadicyclopenta[a,e]pentalene ring. In the aromatic ring which may have a substituent, examples of the type of the substituent which may be included in the aromatic ring include the groups exemplified as the substituent W. In a case where the above-described aromatic ring has a substituent, the number of substituents may be 1 or more (for example, 1 to 4 or the like).

In the present specification, examples of the “aromatic ring group” include a group obtained by removing one or more hydrogen atoms (for example, 1 to 5 or the like) from the above-described aromatic ring.

In the present specification, examples of the “aryl group” include a group obtained by removing one hydrogen atom from a ring corresponding to the aromatic hydrocarbon ring among the above-described aromatic rings.

In the present specification, examples of the “heteroaryl group” include a group obtained by removing one hydrogen atom from a ring corresponding to the aromatic heterocyclic ring among the above-described aromatic rings.

In the present specification, examples of the “arylene group” include a group obtained by removing two hydrogen atoms from a ring corresponding to the aromatic hydrocarbon ring among the above-described aromatic rings.

In the present specification, examples of the “heteroarylene group” include a group obtained by removing two hydrogen atoms from a ring corresponding to the aromatic heterocyclic ring among the above-described aromatic rings.

In the aromatic ring group which may have a substituent, the aryl group which may have a substituent, the heteroaryl group which may have a substituent, the arylene group which may have a substituent, and the heteroarylene group which may have a substituent, examples of the type of the substituent which may be included in these groups include the groups exemplified as the substituent W. In a case where these groups each of which may have a substituent have a substituent, the number of substituents may be 1 or more (for example, 1 to 4 or the like).

In the present specification, the number of ring members in the aliphatic heterocyclic group is preferably 5 to 20, more preferably 5 to 12, and still more preferably 6 to 8. Examples of a heteroatom which is included in the above-described aliphatic heterocyclic group include a sulfur atom, an oxygen atom, a nitrogen atom, a selenium atom, a tellurium atom, a phosphorus atom, a silicon atom, and a boron atom; and a sulfur atom, an oxygen atom, or a nitrogen atom is preferable.

Examples of an aliphatic heterocyclic ring constituting the above-described aliphatic heterocyclic group include a pyrrolidine ring, an oxolane ring, a thiolane ring, a piperidine ring, a tetrahydropyran ring, a thiacine ring, a piperazine ring, a morpholine ring, a quinocyclidine ring, an azetidine ring, an oxetane ring, an aziridine ring, a dioxane ring, and a γ-butyrolactone ring.

### [Photoelectric Conversion Element]

The photoelectric conversion element according to the embodiment of the present invention includes a conductive film, a photoelectric conversion film, and a transparent conductive film in this order, in which the photoelectric conversion film contains a compound represented by Formula (1) described later (hereinafter, referred to as “specific compound”). The mechanism by which the photoelectric conversion element according to the embodiment of the present invention can achieve the above-described problems is not necessarily clear, but the present inventors assume as follows.

The mechanism by which the effect is obtained is not limited by the following supposition. That is, even in a case where the effect is obtained by a mechanism other than the following, it is included in the scope of the present invention.

The present inventors have found that the electric field strength dependence of the response speed is reduced by using a specific compound which is an A-D-A type coloring agent in which an acceptor is directly bonded to a donor having a specific three-fused ring structure. The detailed reason why the above-described effect is obtained is not clear, but it is considered to be due to the selection of the structure of the donor.

It is generally considered that the A-D-A type coloring agent in which an acceptor is directly bonded to a donor having a three-fused ring structure, such as the specific compound according to the present invention, is not desirable for obtaining a high quantum efficiency because the conjugated system is short and thus the absorbance is generally low. However, as a result of a comprehensive search on the A-D-A type coloring agent, the present inventors have found that, contrary to expectation, in a case where the specific compound according to the present invention is used, a high quantum efficiency is obtained, and efficient absorption is exhibited even in a blue and green light region (in the vicinity of 400 to 600 nm). The present inventors presume that the reason why the high quantum efficiency is obtained is that the absorbance of the molecule itself is not so high, but the specific compound has a relatively low molecular weight, and thus, in a case where the specific compound is formed into a film as the photoelectric conversion film, the amount of the substance per unit volume in the film can be increased.

Hereinafter, the fact that the electric field strength dependence of the response speed in a case where the photoelectric conversion element receives blue and green light (light in a wavelength range of 400 to 600 nm) is lower is also referred to as the fact that the effect of the present invention is more excellent.

A configuration of the photoelectric conversion element according to the embodiment of the present invention will be described in detail below.

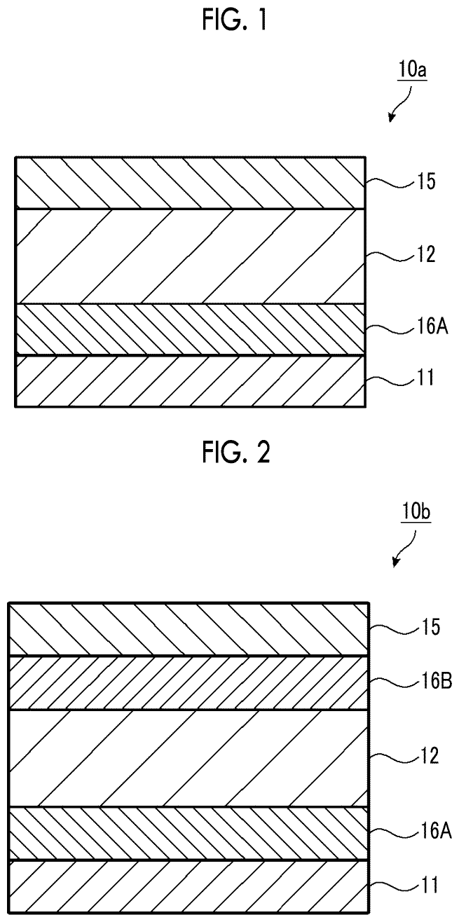

FIG. 1 shows a schematic cross-sectional view of one embodiment of the photoelectric conversion element according to the embodiment of the present invention.

A photoelectric conversion element 10a shown in FIG. 1 has a configuration in which a conductive film (hereinafter, also referred to as “lower electrode”) 11 functioning as a lower electrode, an electron blocking film 16A, a photoelectric conversion film 12 containing the specific compound, and a transparent conductive film (hereinafter, also referred to as “upper electrode”) 15 functioning as an upper electrode are laminated in this order.

FIG. 2 shows a configuration example of another photoelectric conversion element. A photoelectric conversion element 10b shown in FIG. 2 has a configuration in which the electron blocking film 16A, the photoelectric conversion film 12, a hole blocking film 16B, and the upper electrode 15 are laminated on the lower electrode 11 in this order. The lamination order of the electron blocking film 16A, the photoelectric conversion film 12, and the hole blocking film 16B in FIGS. 1 and 2 may be appropriately changed according to the application and the characteristics.

In the photoelectric conversion element 10a (or 10b), it is preferable that light is incident to the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of performance and power consumption, the applied voltage is more preferably 1×10⁻⁴ to 1×10⁷ V/cm and still more preferably 1×10⁻³ to 5×10⁶ V/cm.

Regarding the voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case of being incorporated in an imaging element, the voltage can be applied by the same method.

As described in detail below, the photoelectric conversion element 10a (or 10b) can be suitably applied to applications of an imaging element.

Hereinafter, the form of each layer constituting the photoelectric conversion element according to the embodiment of the present invention will be described in detail.

### [Photoelectric Conversion Film]

The photoelectric conversion element according to the embodiment of the present invention includes a photoelectric conversion film.

### <Specific Compound>

The photoelectric conversion film contains a compound represented by Formula (1) (specific compound).

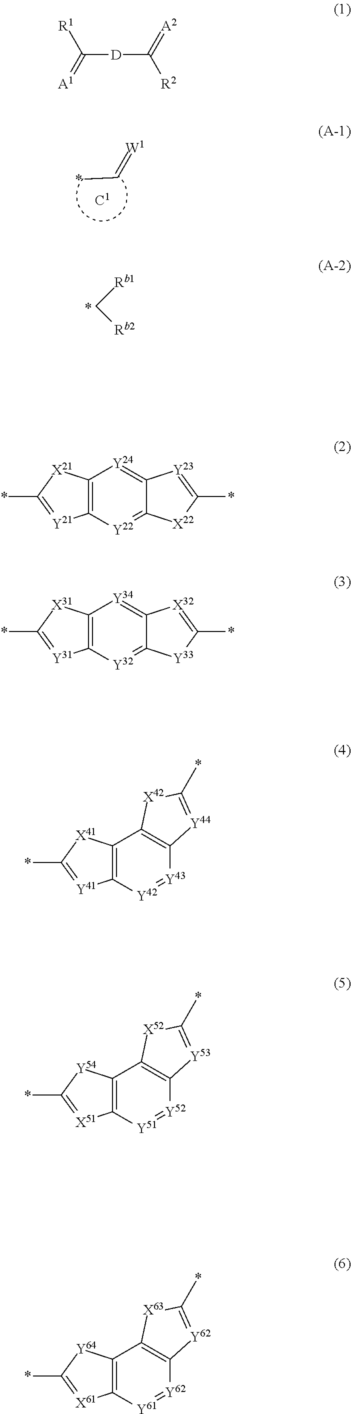

In Formula (1), D represents a group represented by any of Formula (2) to Formula (6).

In Formula (2) to Formula (6), X²¹ represents an oxygen atom, NR^{X1}, or CR^{X2}R^{X3}.

X²², X³¹, X³², X⁴¹, X⁴², X⁵¹, X⁵², X⁶¹, and X⁶² each independently represent a sulfur atom, an oxygen atom, NR^{X1}, or CR^{X2}R^{X3}.

R^{X1} to R^{X3} each independently represent a hydrogen atom, an aliphatic hydrocarbon group which may have a substituent, or an aromatic ring group which may have a substituent.

In Formula (1), from the viewpoint that the effect of the present invention is more excellent, D is preferably the group represented by Formula (2), Formula (4), or Formula (6), and more preferably the group represented by Formula (2).

In Formula (2), X²¹ is preferably an oxygen atom.

In addition, in Formula (2) to Formula (6), X²², X³¹, X³², X⁴¹, X⁴², X⁵¹, X⁵², X⁶¹, and X⁶² are each preferably a sulfur atom or an oxygen atom, and more preferably a sulfur atom.

Examples of the aliphatic hydrocarbon group which may have a substituent, represented by R^{X1} to R^{X3}, include an alkyl group, an alkenyl group, and an alkynyl group. The number of carbon atoms in the above-described aliphatic hydrocarbon group is preferably 1 to 10, more preferably 1 to 6, and still more preferably 1 or 2. Examples of the substituent which may be included in the aliphatic hydrocarbon group include the groups exemplified as the substituent W.

Among these, the aliphatic hydrocarbon group which may have a substituent is preferably a linear or branched alkyl group having 1 to 5 carbon atoms, and more preferably a methyl group, an ethyl group, or an isopropyl group.

In addition, a specific aspect of the aromatic ring group which may have a substituent, represented by R^{X1} to R^{X3}, is as described above; and examples thereof include the aryl group which may have a substituent and the heteroaryl group which may have a substituent. Examples of the substituent which may be included in the aromatic ring group include the groups exemplified as the substituent W. As the substituent which may be included in the aromatic ring group, a methyl group is preferable.

The aromatic ring group which may have a substituent, represented by R^{X1} to R^{X3}, is preferably an aryl group having 6 to 14 carbon atoms, which may have a substituent, or a heteroaryl group having 2 to 14 carbon atoms, which may have a substituent, and more preferably a phenyl group.

In Formula (2) to Formula (6), Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ each independently represent —CR^{Y1}═ or a nitrogen atom. R^{Y1} represents a hydrogen atom or a substituent.

The substituent represented by R^{Y1} is not particularly limited, and examples thereof include the groups exemplified as the substituent W. As the substituent represented by R^{Y1}, a group selected from the following substituent group S is preferable. The preferred aspect of each group in the substituent group S will be described in detail later.

Substituent group S: an alkyl group which may have a substituent, an alkenyl group which may have a substituent, an alkynyl group which may have a substituent, an aryl group which may have a substituent, a heteroaryl group which may have a substituent, an alkoxy group which may have a substituent, an aryloxy group which may have a substituent, an amino group which may have a substituent, a cyano group, and a halogen atom

In Formula (2), it is preferable that at least one of Y²¹, . . . , or Y²⁴ represents —CR^{Y1}═, it is more preferable that at least two thereof represent —CR^{Y1}═, it is still more preferable that at least three thereof represent —CR^{Y1}═, and it is particularly preferable that all of Y²¹ to Y²⁴ represent —CR^{Y1}═.

In Formula (3), it is preferable that at least one of Y³¹, . . . , or Y³⁴ represents —CR^{Y1}═, it is more preferable that at least two thereof represent —CR^{Y1}═, it is still more preferable that at least three thereof represent —CR^{Y1}=, and it is particularly preferable that all of Y³¹ to Y³⁴ represent —CR^{Y1}═.

In Formula (4), it is preferable that at least one of Y⁴¹, . . . , or Y⁴¹ represents —CR^{Y1}═, it is more preferable that at least two thereof represent —CR^{Y1}═, it is still more preferable that at least three thereof represent —CR^{Y1}═, and it is particularly preferable that all of Y⁴¹ to Y⁴⁴ represent —CR^{Y1}═.

In Formula (5), it is preferable that at least one of Y⁵¹, . . . , or Y⁵⁴ represents —CR^{Y1}═, it is more preferable that at least two thereof represent —CR^{Y1}═, it is still more preferable that at least three thereof represent —CR^{Y1}═, and it is particularly preferable that all of Y⁵¹ to Y⁵⁴ represent —CR^{Y1}═.

In Formula (6), it is preferable that at least one of Y⁶¹, . . . , or Y⁶⁴ represents —CR^{Y1}=, it is more preferable that at least two thereof represent —CR^{Y1}=, it is still more preferable that at least three thereof represent —CR^{Y1}=, and it is particularly preferable that all of Y⁶¹ to Y⁶⁴ represent —CR^{Y1}═.

In Formula (2) to Formula (6), a plurality of R^{Y1}'s may be the same or different from each other.

In Formula (2), it is preferable that at least one of Y²¹, . . . , or Y²⁴ represents —CRs=, it is preferable that one or two thereof represent —CRs=, and it is more preferable that one thereof represents —CRs=.

In Formula (3), it is preferable that at least one of Y³¹, . . . , or Y³⁴ represents —CRs=, it is preferable that one or two thereof represent —CRs=, and it is more preferable that one thereof represents —CRs=.

In Formula (4), it is preferable that at least one of Y⁴¹, . . . , or Y⁴⁴ represents —CRs=, it is preferable that one or two thereof represent —CRs=, and it is more preferable that one thereof represents —CRs=.

In Formula (5), it is preferable that at least one of Y⁵¹, . . . , or Y⁵⁴ represents —CRs=, it is preferable that one or two thereof represent —CRs=, and it is more preferable that one thereof represents —CRs=.

In Formula (6), it is preferable that at least one of Y⁶¹, . . . , or Y⁶⁴ represents —CRs=, it is preferable that one or two thereof represent —CRs=, and it is more preferable that one thereof represents —CRs=.

In Formula (2) to Formula (6), in a case where a plurality of Rs's are present, Rs's may be the same or different from each other, but it is preferable that Rs's represent groups different from each other.

For the calculation of the number of —CRs='s as described above, in a case where the group represented by Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ can correspond to both —CR^{Y1}═ and —CRs=, the group is counted as —CRs=. For example, in a case where two of Y⁴¹ and Y⁴² are —CCH₃=, —CCH₃=corresponds to both —CR^{Y1}═ and CRs=, but the count is made such that there are two —CRs═ in Formula (4). In other words, a method of counting such that one of two —CCH₃='s corresponds to —CR^{Y1}═ and the other corresponds to —CRs=, and thus the number of —CRs═ is one is not used.

Rs's each independently represent a group selected from the substituent group S.

Substituent group S: an alkyl group which may have a substituent, an alkenyl group which may have a substituent, an alkynyl group which may have a substituent, an aryl group which may have a substituent, a heteroaryl group which may have a substituent, an alkoxy group which may have a substituent, an aryloxy group which may have a substituent, an amino group which may have a substituent, a cyano group, and a halogen atom

Among these, Rs is preferably an alkyl group which may have a substituent, an alkoxy group which may have a substituent, an aryl group which may have a substituent, a heteroaryl group which may have a substituent, or a halogen atom; more preferably an alkyl group which may have a substituent, an aryl group which may have a substituent, a heteroaryl group which may have a substituent, or a halogen atom; still more preferably an alkyl group which may have a substituent, an aryl group which may have a substituent, or a halogen atom; and particularly preferably an alkyl group which may have a substituent or an aryl group which may have a substituent.

As described above, in a case where a plurality of Rs's are present, Rs's may be the same or different from each other, but it is preferable that Rs's represent groups different from each other.

The above-described alkyl group which may have a substituent may be linear, branched, or cyclic.

In a case where the above-described alkyl group is a linear alkyl group, the number of carbon atoms is preferably 1 to 10, more preferably 1 to 5, and still more preferably 1 or 2.

In a case where the above-described alkyl group is a branched alkyl group, the number of carbon atoms is preferably 3 to 10, more preferably 3 to 7, and still more preferably 3 or 4.

In a case where the above-described alkyl group is a cyclic alkyl group, the number of carbon atoms is preferably 3 to 10 and more preferably 3 to 6.

In addition, examples of the substituent which may be included in the alkyl group include the groups exemplified as the substituent W. As the substituent which may be included in the alkyl group, a halogen atom is preferable, and a fluorine atom is more preferable. The alkyl group may be a perfluoroalkyl group such as a trifluoromethyl group.

Among these, a methyl group or an isopropyl group is preferable as the alkyl group.

The alkenyl group which may have a substituent, represented by Rs, may be linear, branched, or cyclic.

The number of carbon atoms in the alkenyl group is preferably 2 to 10, more preferably 2 to 6, and still more preferably 2 to 5.

In addition, examples of the substituent which may be included in the alkenyl group include the groups exemplified as the substituent W. As the substituent which may be included in the alkenyl group, an aryl group is preferable, an aryl group having 6 to 10 carbon atoms is more preferable, and a phenyl group is still more preferable.

Among these, a vinyl group, a group obtained by removing a hydrogen atom at the 2-position of propylene, a group obtained by removing a vinyl hydrogen atom at a terminal of styrene, or a group obtained by removing a hydrogen atom at the 1-position of isobutene is preferable as the alkenyl group.

Examples of the substituent which may be included in the above-described alkynyl group which may have a substituent include the groups exemplified as the substituent W. As the substituent which may be included in the alkynyl group, an aryl group having 6 to 10 carbon atoms is preferable, and a phenyl group is more preferable.

The number of carbon atoms in the alkynyl group is preferably 2 to 10, more preferably 2 to 6, and still more preferably 2 to 5.

Among these, the alkynyl group which may have a substituent is preferably a propargyl group, a 1-methylpropargyl group, or a phenylacetynyl group.

The above-described aryl group which may have a substituent is not particularly limited, and may be monocyclic or polycyclic.

The number of carbon atoms in the above-described aryl group is preferably 6 to 20, more preferably 6 to 14, and still more preferably 6 to 10. Among these, a phenyl group is preferable as the aryl group.

Examples of the substituent which may be included in the aryl group include the groups exemplified as the substituent W. Examples of the substituent which may be included in the aryl group include a halogen atom, an alkyl group, and an alkoxy group. Specific aspects of the above-described groups are as described above, but the substituent which may be included in the aryl group is preferably a methyl group, an isopropyl group, or a methoxy group.

The above-described heteroaryl group which may have a substituent is not particularly limited, and may be monocyclic or polycyclic.

Specific examples of an aromatic heterocyclic ring constituting the above-described heteroaryl group are as described above.

The number of carbon atoms in the heteroaryl group is preferably 2 to 20, more preferably 2 to 14, and still more preferably 2 to 10. In addition, the number of heteroatoms included in the heteroaryl group is preferably 1 to 10, more preferably 1 to 3, and still more preferably 1 or 2. Among these, a group obtained by removing a hydrogen atom from the 2-position of thiophene is preferable as the heteroaryl group.

Examples of the substituent which may be included in the heteroaryl group include the groups exemplified as the substituent W. Examples of the substituent which may be included in the heteroaryl group include a halogen atom, an alkyl group, and an alkoxy group. Specific aspects of the above-described groups are as described above, but the substituent which may be included in the heteroaryl group is preferably a methyl group, an isopropyl group, or a methoxy group.

In the above-described alkoxy group which may have a substituent, an alkyl group bonded to an oxygen atom may be linear, branched, or cyclic.

In a case where the above-described alkyl group is a linear alkyl group, the number of carbon atoms is preferably 1 to 10, more preferably 1 to 5, and still more preferably 1 or 2.

In a case where the above-described alkyl group is a branched alkyl group, the number of carbon atoms is preferably 3 to 10 and more preferably 3 to 7.

In a case where the above-described alkyl group is a cyclic alkyl group, the number of carbon atoms is preferably 3 to 10 and more preferably 3 to 6.

Examples of the substituent which may be included in the alkoxy group include the groups exemplified as the substituent W.

In the above-described aryloxy group which may have a substituent, an aryl group bonded to an oxygen atom is not particularly limited, and may be monocyclic or polycyclic.

The number of carbon atoms in the above-described aryloxy group is preferably 6 to 20, more preferably 6 to 14, and still more preferably 6 to 10.

Examples of the substituent which may be included in the aryloxy group include the groups exemplified as the substituent W. Examples of the substituent which may be included in the aryl group include a halogen atom, an alkyl group, and an alkoxy group. Specific aspects of the above-described groups are as described above, but the substituent which may be included in the aryloxy group is preferably a methyl group, an isopropyl group, or a methoxy group.

The above-described amino group which may have a substituent is not particularly limited, and may be a primary amino group (—NH₂), a secondary amino group (—NR^{T}H), or a tertiary amino group (—NR^{T}₂).

R^{T} represents a substituent which may be included in the above-described amino group, and a plurality of R^{T}'s may be different from each other. R^{T} is preferably the above-described alkyl group which may have a substituent, and more preferably an alkyl group having 1 to 3 carbon atoms.

As the halogen atom, a fluorine atom or a chlorine atom is preferable.

From the viewpoint that the effect of the present invention is more excellent, it is preferable that D in Formula (1) represents a group represented by any of Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), or Formula (6-12).

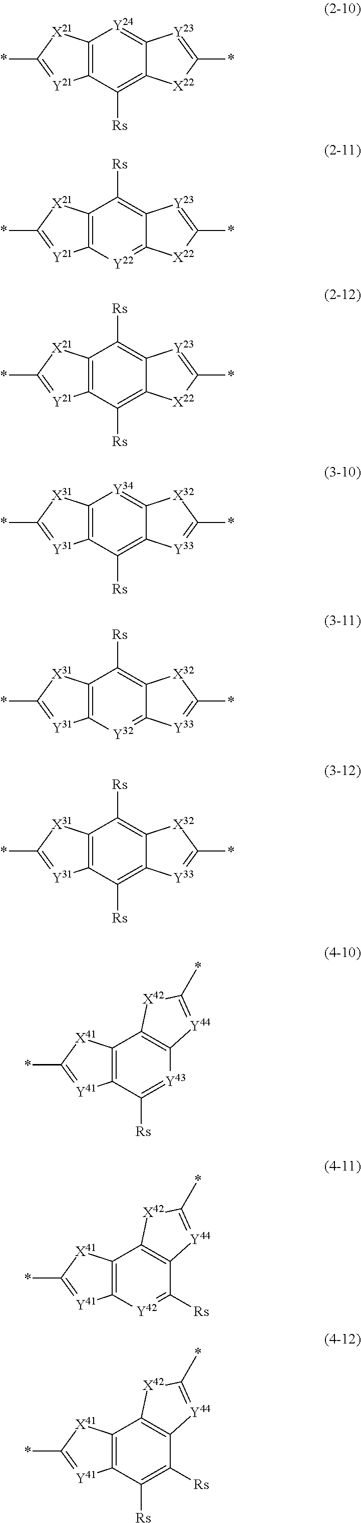

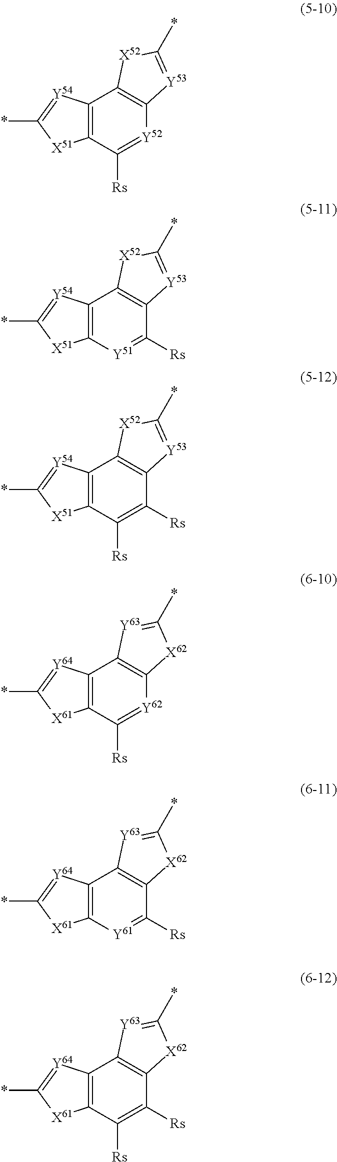

In Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), and Formula (6-12), X²¹ represents an oxygen atom, NR^{X1}, or CR^{X2}R^{X3}.

X²², X³¹, X³², X⁴¹, X⁴², X⁵¹, X⁵², X⁶¹, and X⁶² each independently represent a sulfur atom, an oxygen atom, NR^{X1}, or CR^{X2}R^{X3}.

R^{X1} to R^{X3} each independently represent a hydrogen atom, an aliphatic hydrocarbon group which may have a substituent, or an aromatic ring group which may have a substituent.

Specific aspects and preferred aspects of R^{X1} to R^{X3} are as described above.

Specific aspects and preferred aspects of X²², X³¹, X³², X⁴¹, X⁴², X⁵¹, X⁵², X⁶¹, and X⁶² are the same as the specific aspects and preferred aspects of X²², X³¹, X³², X⁴¹, X⁴², X⁵¹, X⁵², X⁶¹, and X⁶² in Formula (2) to Formula (6).

In Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), and Formula (6-12), Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ each independently represent —CR^{Y1}═ or a nitrogen atom. R^{Y1} represents a hydrogen atom or a substituent.

Specific aspects and suitable aspects of Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ are the same as the specific aspects and preferred aspects of Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ in Formula (2) to Formula (5).

In Formula (2-10), it is preferable that at least one of Y²¹, Y²³, or Y²⁴ represents —CR^{Y1}=, it is more preferable that at least two thereof represent —CR^{Y1}=, and it is still more preferable that all of Y²¹, Y²³, and Y²⁴ represent —CR^{Y1}═.

In Formula (2-11), it is preferable that at least one of Y²¹, . . . , or Y²³ represents —CR^{Y1}=, it is more preferable that at least two thereof represent —CR^{Y1}═, and it is still more preferable that all of Y²¹ to Y²³ represent —CR^{Y1}═.

In Formula (2-12), it is preferable that at least one of Y²¹ or Y²³ represents —CR^{Y1}═, and it is more preferable that both Y²¹ and Y²³ represent —CR^{Y1}=.

In Formula (3-10), it is preferable that at least one of Y³¹, Y³³, or Y³⁴ represents —CR^{Y1}=, it is more preferable that at least two thereof represent —CR^{Y1}=, and it is still more preferable that all of Y³¹, Y³³, and Y³⁴ represent —CR^{Y1}=.

In Formula (3-11), it is preferable that at least one of Y³¹, . . . , or Y³³ represents —CR^{Y1}=, it is more preferable that at least two thereof represent —CR^{Y1}=, and it is still more preferable that all of Y³¹ to Y³³ represent —CR^{Y1}=.

In Formula (3-12), it is preferable that at least one of Y³¹ or Y³³ represents —CR^{Y1}=, and it is more preferable that both Y³¹ and Y³³ represent —CR^{Y1}=.

In Formula (4-10), it is preferable that at least one of Y⁴¹, Y⁴³, or Y⁴⁴ represents —CR^{Y1}=, it is more preferable that at least two thereof represent —CR^{Y1}=, and it is still more preferable that all of Y⁴¹, Y⁴³, and Y⁴⁴ represent —CR^{Y1}=.

In Formula (4-11), it is preferable that at least one of Y⁴¹, Y⁴², or Y⁴⁴ represents —CR^{Y1}=, it is more preferable that at least two thereof represent —CR^{Y1}=, and it is still more preferable that all of Y⁴¹, Y⁴², and Y⁴⁴ represent —CR^{Y1}=.

In Formula (4-12), it is preferable that at least one of Y⁴¹ or Y⁴⁴ represents —CR^{Y1}=, and it is more preferable that both Y⁴¹ and Y⁴⁴ represent —CR^{Y1}=.

In Formula (5-10), it is preferable that at least one of Y⁵², . . . , or Y⁵⁴ represents —CR^{Y1}=, it is more preferable that at least two thereof represent —CR^{Y1}=, and it is still more preferable that all of Y⁵² to Y⁵⁴ represent —CR^{Y1}=.

In Formula (5-11), it is preferable that at least one of Y⁵¹, Y⁵³, or Y⁵⁴ represents —CR^{Y1}=, it is more preferable that at least two thereof represent —CR^{Y1}=, and it is still more preferable that all of Y⁵¹, Y⁵³, and Y⁵⁴ represent —CR^{Y1}=.

In Formula (5-12), it is preferable that at least one of Y⁵³ or Y⁵⁴ represents —CR^{Y1}=, and it is more preferable that both Y⁵³ and Y⁵⁴ represent —CR^{Y1}=.

In Formula (6-10), it is preferable that at least one of Y⁶², . . . , or Y⁶⁴ represents —CR^{Y1}=, it is more preferable that at least two thereof represent —CR^{Y1}=, and it is still more preferable that all of Y⁶² to Y⁶⁴ represent —CR^{Y1}=.

In Formula (6-11), it is preferable that at least one of Y⁶¹, Y⁶³, or Y⁶⁴ represents —CR^{Y1}=, it is more preferable that at least two thereof represent —CR^{Y1}=, and it is still more preferable that all of Y⁶¹, Y⁶³, and Y⁶⁴ represent —CR^{Y1}=.

In Formula (6-12), it is preferable that at least one of Y⁶³ or Y⁶⁴ represents —CR^{Y1}=, and it is more preferable that both Y⁶³ and Y⁶⁴ represent —CR^{Y1}=.

In Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), and Formula (6-12), Rs's each independently represent a group selected from the above-described substituent group S.

Specific aspects and suitable aspects of each group exemplified as the substituent group S are as described above.

Among the above, Rs is preferably an alkyl group which may have a substituent, an alkoxy group which may have a substituent, an aryl group which may have a substituent, a heteroaryl group which may have a substituent, or a halogen atom; more preferably an alkyl group which may have a substituent, an aryl group which may have a substituent, a heteroaryl group which may have a substituent, or a halogen atom; still more preferably an alkyl group which may have a substituent, an aryl group which may have a substituent, or a halogen atom; and particularly preferably an alkyl group which may have a substituent or an aryl group which may have a substituent.

In Formula (2-12), Formula (3-12), Formula (4-12), Formula (5-12), and Formula (6-12), in which two Rs's are present, the two Rs's may be the same group or different groups from each other, but are preferably different groups from each other.

In Formula (1), R¹ and R² each independently represent a hydrogen atom or a substituent.

Examples of the substituent represented by R¹ and R² include the groups exemplified as the substituent W. Among these, from the viewpoint that the effect of the present invention is more excellent, R¹ and R² are each preferably a hydrogen atom.

In Formula (1), A¹ and A² each independently represent the above-described group represented by Formula (A-1) or the above-described group represented by Formula (A-2).

In Formula (A-1), W¹ represents a sulfur atom, an oxygen atom, =NR^{W2}, or =CR^{W3}R^{W4}.

R^{W2} represents a hydrogen atom or a substituent. R^{W3} and R^{W4} each independently represent a cyano group, —SO₂R^{W5}, —COOR^{W6}, or —COR^{W7}.

From the viewpoint that the effect of the present invention is more excellent, W¹ is preferably an oxygen atom or a sulfur atom.

Examples of the substituent represented by R^{W2} include the substituents exemplified as the substituent W.

In addition, R^{W5} to R^{W7} each independently represent an aliphatic hydrocarbon group which may have a substituent, an aromatic ring group which may have a substituent, or an aliphatic heterocyclic group which may have a substituent.

Examples of the substituent which may be included in the groups represented by R^{W5} to R^{W7} include the substituents exemplified as the substituent W.

The definition of the above-described aliphatic hydrocarbon group is as described above; and among these, an alkyl group is preferable, and a linear alkyl group is more preferable. The number of carbon atoms in the aliphatic hydrocarbon group is preferably 1 to 3.

The definition of the above-described aromatic ring group is as described above; and among these, an aryl group is preferable, and a phenyl group is more preferable.

The definition of the above-descried aliphatic heterocyclic group is as described above.

In Formula (A-1), C¹ represents a ring which contains two or more carbon atoms and may have a substituent.

The number of carbon atoms in the above-described ring is preferably 3 to 30, more preferably 3 to 20, and still more preferably 3 to 10. The above-described number of carbon atoms is a number including two carbon atoms specified in the formula.

The above-described ring may be aromatic or non-aromatic.

The above-described ring may be any of a monocyclic ring or a polycyclic ring, and is preferably a 5-membered ring, a 6-membered ring, or a fused ring including at least one of a 5-membered ring or a 6-membered ring. The number of rings forming the fused ring is preferably 1 to 4 and more preferably 1 to 3.

The above-described ring may have a heteroatom. Examples of the heteroatom include a nitrogen atom, a sulfur atom, an oxygen atom, a selenium atom, a tellurium atom, a phosphorus atom, a silicon atom, and a boron atom; and a sulfur atom, a nitrogen atom, or an oxygen atom is preferable.

The number of heteroatoms in the above-described ring is preferably 0 to 10 and more preferably 0 to 5.

Among the carbon atoms constituting the ring represented by C¹, a carbon atom other than the carbon atom at a bonding position to which * is attached in Formula (A-1) and the carbon atom bonded to W¹ may be replaced with a carbonyl carbon (>C=O) or a thiocarbonyl carbon (>C=S).

Examples of the substituent which may be included in the ring include the groups exemplified as the substituent W; and a halogen atom, an alkyl group, an aromatic ring group, or a silyl group is preferable, and a halogen atom or an alkyl group is more preferable.

The above-described alkyl group may be linear, branched, or cyclic, and is preferably linear.

The number of carbon atoms in the above-described alkyl group is preferably 1 to 10 and more preferably 1 to 3.

In Formula (A-1), as the above-described ring represented by C¹, a ring used as an acidic nucleus (for example, an acidic nucleus of a merocyanine coloring agent) is preferable; and examples thereof include the following nuclei.
(a) 1,3-dicarbonyl nucleus: for example, 1,3-indandione nucleus, 1,3-cyclohexanedione, 5,5-dimethyl-1,3-cyclohexanedione, 1,3-dioxane-4,6-dione, and the like
(b) pyrazolinone nucleus: for example, 1-phenyl-2-pyrazolin-5-one, 3-methyl-1-phenyl-2-pyrazolin-5-one, 1-(2-benzothiazolyl)-3-methyl-2-pyrazolin-5-one, and the like
(c) isoxazolinone nucleus: for example, 3-phenyl-2-isoxazolin-5-one, 3-methyl-2-isoxazolin-5-one, and the like
(d) oxindole nucleus: for example, 1-alkyl-2,3-dihydro-2-oxindole and the like
(e) 2,4,6-trioxohexahydropyrimidine nucleus: for example, barbituric acid, 2-thibarbituric acid and derivatives thereof, and the like; examples of the above-described derivatives include a 1-alkyl form such as 1-methyl and 1-ethyl, a 1,3-dialkyl form such as 1,3-dimethyl, 1,3-diethyl, and 1,3-dibutyl, a 1,3-diaryl form such as 1,3-diphenyl, 1,3-di(p-chlorophenyl), 1,3-di(p-ethoxycarbonylphenyl), a 1-alkyl-1-aryl form such as 1-ethyl-3-phenyl, and a 1,3-diheteroaryl form such as 1,3-di(2-pyridyl).
(f) 2-thio-2,4-thiazolidinedione nucleus: for example, rhodanine and derivatives thereof, and the like; examples of the above-described derivatives include a 3-alkylrhodanine such as 3-methylrhodanine, 3-ethylrhodanine, and 3-allylrhodanine, a 3-arylrhodanine such as 3-phenylrhodanine, and a 3-heteroaryl rhodanine such as 3-(2-pyridyl)rhodanine.
(g) 2-thio-2,4-oxazolidinedione (2-thio-2,4-(3H,5H)-oxazoledione) nucleus: for example, 3-ethyl-2-thio-2,4-oxazolidinedione and the like
(h) thianaphthenone nucleus: for example, 3(2H)-thianaphthenone-1,1-dioxide and the like
(i) 2-thio-2,5-thiazolidinedione nucleus: for example, 3-ethyl-2-thio-2,5-thiazolidinedione and the like
(j) 2,4-thiazolidinedione nucleus: for example, 2,4-thiazolidinedione, 3-ethyl-2,4-thiazolidinedione, 3-phenyl-2,4-thiazolidinedione, and the like
(k) thiazolin-4-one nucleus: for example, 4-thiazolinone, 2-ethyl-4-thiazolinone, and the like
(l) 2,4-imidazolidinedione (hydantoin) nucleus: for example, 2,4-imidazolidinedione, 3-ethyl-2,4-imidazolidinedione, and the like
(m) 2-thio-2,4-imidazolidinedione (2-thiohydantoine) nucleus: for example, 2-thio-2,4-imidazolidinedione, 3-ethyl-2-thio-2,4-imidazolidinedione, and the like
(n) imidazolin-5-one nucleus: for example, 2-propylmercapto-2-imidazolin-5-one and the like
(o) 3,5-pyrazolidinedione nucleus: for example, 1,2-diphenyl-3,5-pyrazolidinedione, 1,2-dimethyl-3,5-pyrazolidinedione, and the like
(p) benzothiophen-3(2H)-one nucleus: for example, benzothiophen-3(2H)-one, oxobenzothiophen-3(2H)-one, dioxobenzothiophen-3(2H)-one, and the like
(q) indanone nucleus: for example, 1-indanone, 3-phenyl-1-indanone, 3-methyl-1-indanone, 3,3-diphenyl-1-indanone, 3,3-dimethyl-1-indanone, and the like
(r) benzofuran-3-(2H)-one nucleus: for example, benzofuran-3-(2H)-one and the like
(s) 2,2-dihydrophenalene-1,3-dione nucleus and the like

From the viewpoint that the effect of the present invention is more excellent, the above-described group represented by Formula (A-1) is preferably a group represented by Formula (A-11).

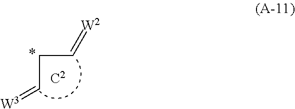

In Formula (A-11), * represents a bonding position.

W² and W³ each independently represent an oxygen atom or a sulfur atom.

It is preferable that both W² and W³ represent an oxygen atom.

In addition, in Formula (A-11), C² represents a ring which contains three or more carbon atoms and may have a substituent.

Three carbon atoms contained in C² are the three carbon atoms specified in Formula (A-11).

The number of carbon atoms in the above-described ring is preferably 3 to 30, more preferably 3 to 20, and still more preferably 3 to 10. The number of carbon atoms in the above-described ring is the number including the three carbon atoms specified in the formula.

The above-described ring may be an aromatic ring or a non-aromatic ring.

The above-described ring may be any of a monocyclic ring or a polycyclic ring, and is preferably a 5-membered ring, a 6-membered ring, or a fused ring including at least one of a 5-membered ring or a 6-membered ring. In a case where the ring is a polycyclic ring, the number of rings included is preferably 2 to 6 and more preferably 2 or 3.

The above-described ring may have a heteroatom. Examples of the heteroatom include a nitrogen atom, a sulfur atom, an oxygen atom, a selenium atom, a tellurium atom, a phosphorus atom, a silicon atom, and a boron atom; and a sulfur atom, a nitrogen atom, or an oxygen atom is preferable.

The number of heteroatoms in the above-described ring is preferably 0 to 10 and more preferably 0 to 5.

Among the carbon atoms constituting the ring represented by C², a carbon atom other than the carbon atom at a bonding position to which * is attached in Formula (A-11) and the carbon atom bonded to W² and W³ may be replaced with a carbonyl carbon (>C═O) or a thiocarbonyl carbon (>C=S).

A suitable aspect of the substituent which may be included in the ring is the same as that of the substituent which may be included in the ring C¹ described above.

In addition, it is preferable that the above-described group represented by Formula (A-11) is a group represented by Formula (C-1) or a group represented by Formula (C-2).

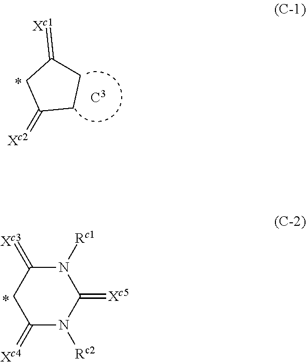

In Formula (C-1), X^{c1} and X^{c2} each independently represent a sulfur atom or an oxygen atom.

It is preferable that at least one of X¹ or X^{c2} is an oxygen atom, and it is more preferable that both X^{c1} and X^{c2} are oxygen atoms.

In Formula (C-1), C³ represents an aromatic ring which may have a substituent. The number of carbon atoms in the above-described aromatic ring is preferably 4 to 30, more preferably 5 to 12, and still more preferably 6 to 8. The above-described number of carbon atoms is a number including two carbon atoms specified in the formula.

The above-described aromatic ring may be monocyclic or polycyclic.

In addition, the aromatic ring may be any of an aromatic hydrocarbon ring or an aromatic heterocyclic ring, and an aromatic hydrocarbon ring is preferable.

Examples of the aromatic ring represented by C³ include the rings exemplified in the description of the aromatic ring described above.

Among these, as the aromatic ring represented by C³, a benzene ring, a naphthalene ring, an anthracene ring, or a pyrene ring is preferable, and a benzene ring is more preferable. Examples of the substituent which may be included in the aromatic ring include the groups exemplified as the substituent W.

In Formula (C-2), X^{c3} to X^{c5} represent a sulfur atom or an oxygen atom.

It is preferable that both X^{c3} and X⁴ are oxygen atoms, and it is more preferable that all of X^{c3}, X¹⁴, and X^{c5} are oxygen atoms.

In addition, R^{c1} and R^{c2} each independently represent a hydrogen atom or a substituent. Examples of the substituent represented by R^{c1} and R^{c2} include the groups exemplified as the substituent W, and among these, an alkyl group or a phenyl group is preferable, and an alkyl group is more preferable.

The above-described phenyl group may further have a substituent, and examples thereof include the groups exemplified as the substituent W.

In Formula (A-2), R^{b1} and R^{b2} each independently represent a cyano group, —COOR^{b3}, or —COR^{b4}, and a cyano group or —COOR^{b3} is preferable.

R^{b3} and R^{b4} each independently represent an aliphatic hydrocarbon group which may have a substituent, an aromatic ring group which may have a substituent, or an aliphatic heterocyclic group which may have a substituent.

The definition and suitable aspect of each group represented by R^{b3} and R^{b4} are the same as those of each group represented by R^{W5} to R^{W7}; and among these, an aliphatic hydrocarbon group having 1 to 4 carbon atoms or a phenyl group is preferable.

From the viewpoint that the quantum efficiency, the response speed, and the effect of the present invention are more excellent, A¹ and A² are preferably the group represented by Formula (A-1), more preferably the group represented by Formula (A-11), still more preferably the group represented by Formula (C-1) or the group represented by Formula (C-2), and particularly preferably the group represented by Formula (C-2).

In addition, A¹ and A² may be the same or different from each other, but are preferably the same.

A molecular weight of the specific compound is preferably 300 to 1,200, more preferably 350 to 1,000, and still more preferably 400 to 800.

In a case where the molecular weight is in the above-described range, it is presumed that sublimation temperature of the specific compound is low, and thus the quantum efficiency is excellent also in a case where the photoelectric conversion film is formed at a high speed.

From the viewpoint of stability in a case of using the specific compound as a p-type organic semiconductor and matching of energy levels between the specific compound and an n-type organic semiconductor, an ionization potential of the specific compound in a single film is preferably −6.0 to −5.0 eV.

A maximal absorption wavelength of the specific compound is preferably in a wavelength range of 400 to 600 nm, and more preferably in a wavelength range of 400 to 500 nm.

The above-described maximal absorption wavelength is a value measured in a solution state (solvent:chloroform) by adjusting the absorption spectrum of the specific compound to a concentration such that the light absorbance is 0.5 to 1.0. However, in a case where the specific compound is not soluble in chloroform, a value measured by using the specific compound in which the specific compound is vapor-deposited and formed into a film state is defined as the maximal absorption wavelength of the specific compound.

The specific compound is particularly useful as a material of a photoelectric conversion film used for an imaging element, an optical sensor, or a photoelectric cell. The specific compound usually functions as a coloring agent in the photoelectric conversion film. In addition, the specific compound can also be used as a coloring material, a liquid crystal material, an organic semiconductor material, a charge transport material, a pharmaceutical material, and a fluorescent diagnostic material.

Hereinafter, specific examples of D (the group represented by any of Formula (2) to Formula (6)) in the compound represented by Formula (1) (specific compound) are shown below, but the present invention is not limited thereto.

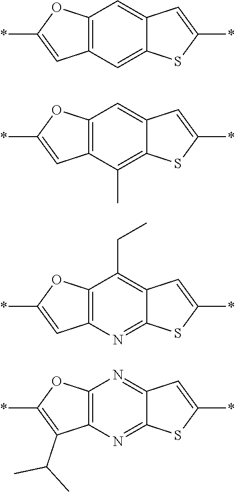

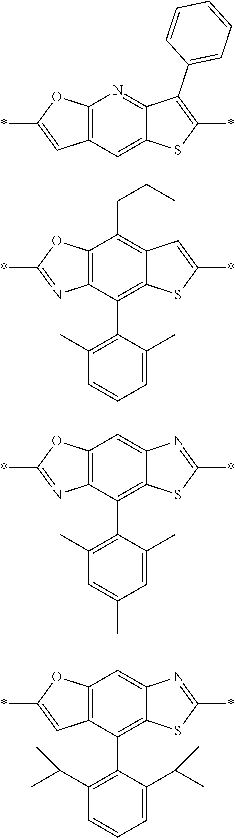

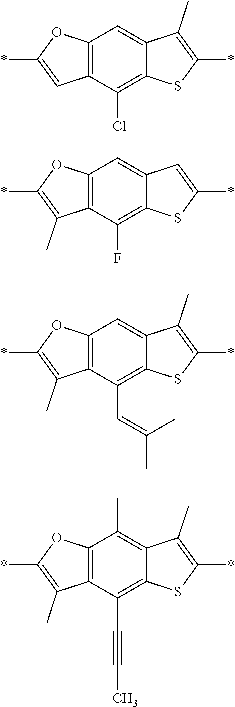

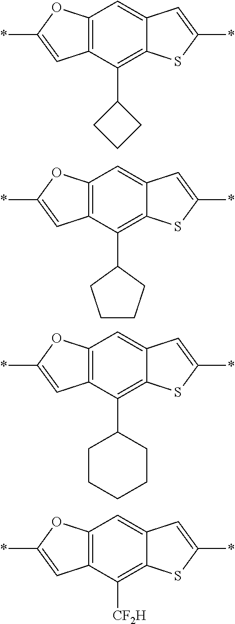

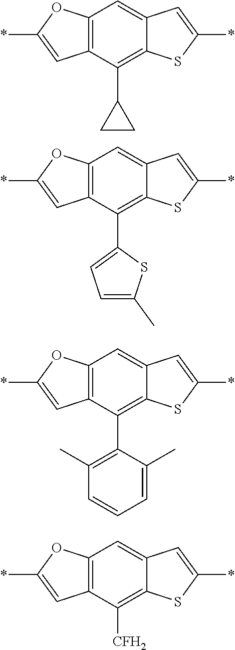

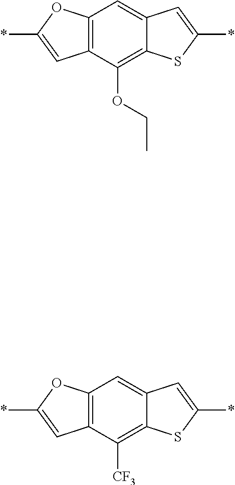 [SMILES: *c1cc2c(OCC)c3sc(*)cc3cc2o1 ; *c1cc2c(C(F)(F)F)c3sc(*)cc3cc2o1]

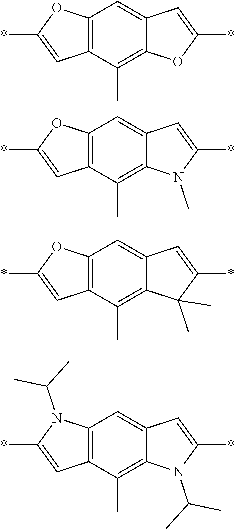

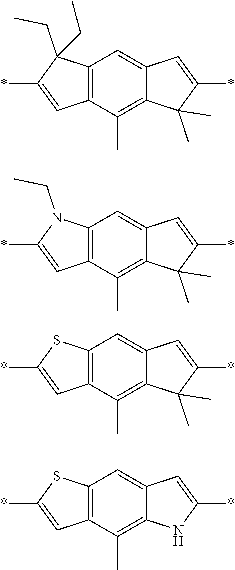

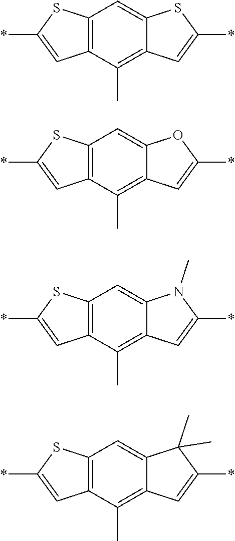

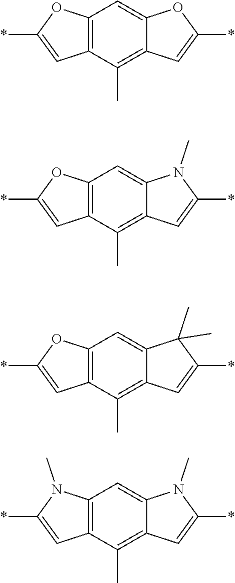 [SMILES: *c1cc2c(C)c3cc(*)oc3cc2o1 ; *c1cc2c(C)c3cc(*)n(C)c3cc2o1 ; *C1=Cc2c(cc3oc(*)cc3c2C)C1(C)C ; *c1cc2c(C)c3cc(*)n(C)c3cc2n1C]

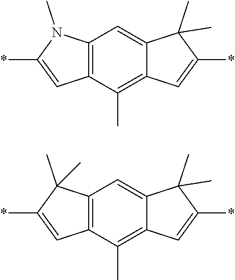 [SMILES: *C1=Cc2c(cc3c(cc(*)n3C)c2C)C1(C)C ; *C1=Cc2c(cc3c(c2C)C=C(*)C3(C)C)C1(C)C]

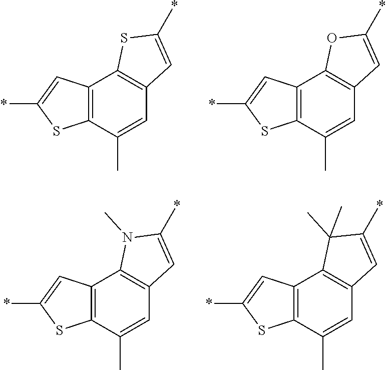

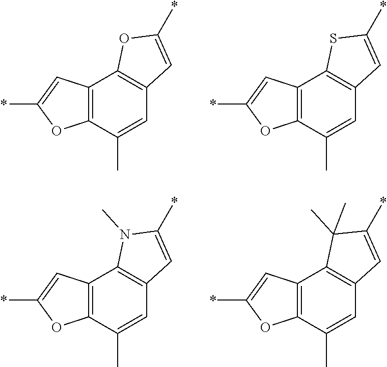

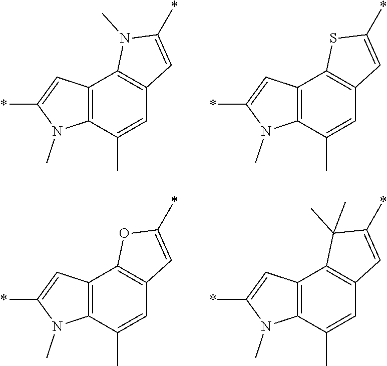

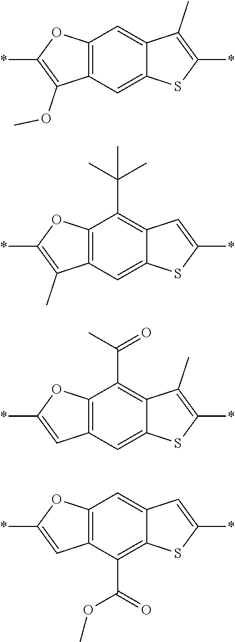

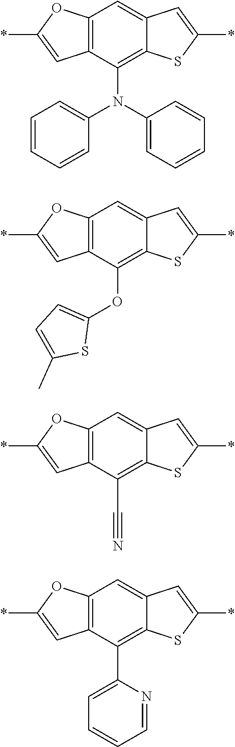

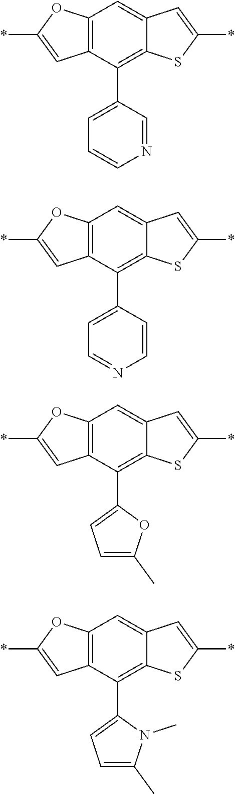

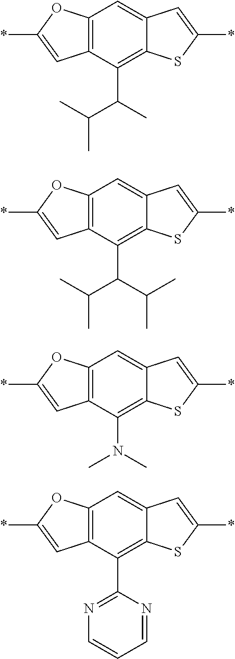

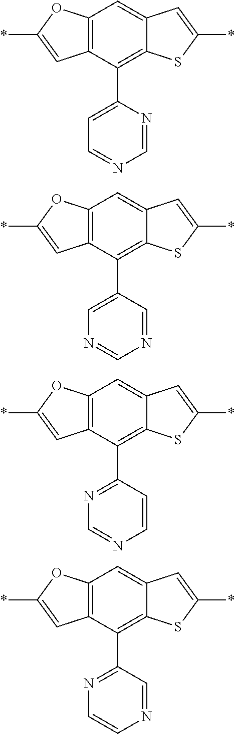

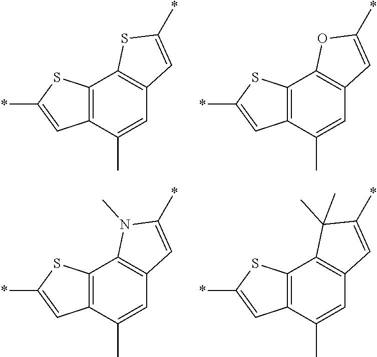

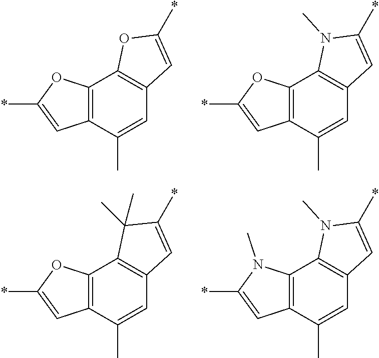

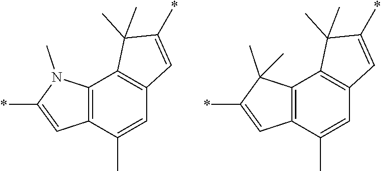 [SMILES: *C1=Cc2cc(C)c3cc(*)n(C)c3c2C1(C)C ; *C1=Cc2cc(C)c3c(c2C1(C)C)C(C)(C)C(*)=C3]

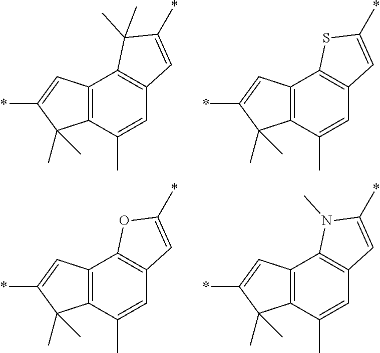

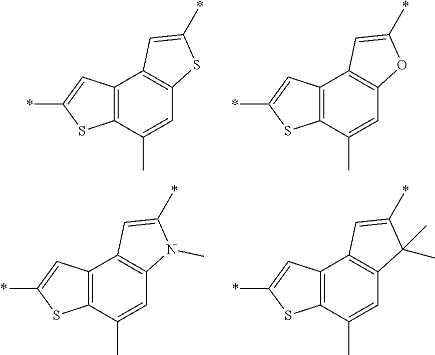

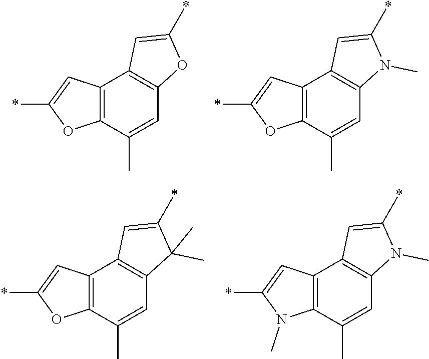

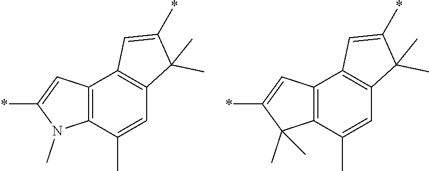

In addition, specific examples of A¹ and A² in the compound represented by Formula (1) (specific compound) are shown below, but the present invention is not limited thereto.

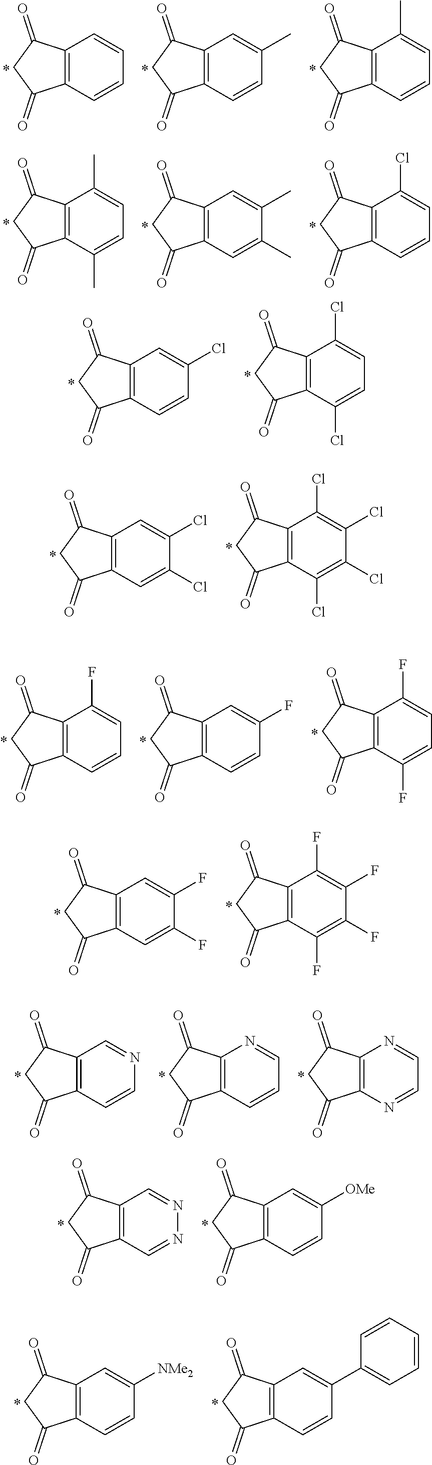

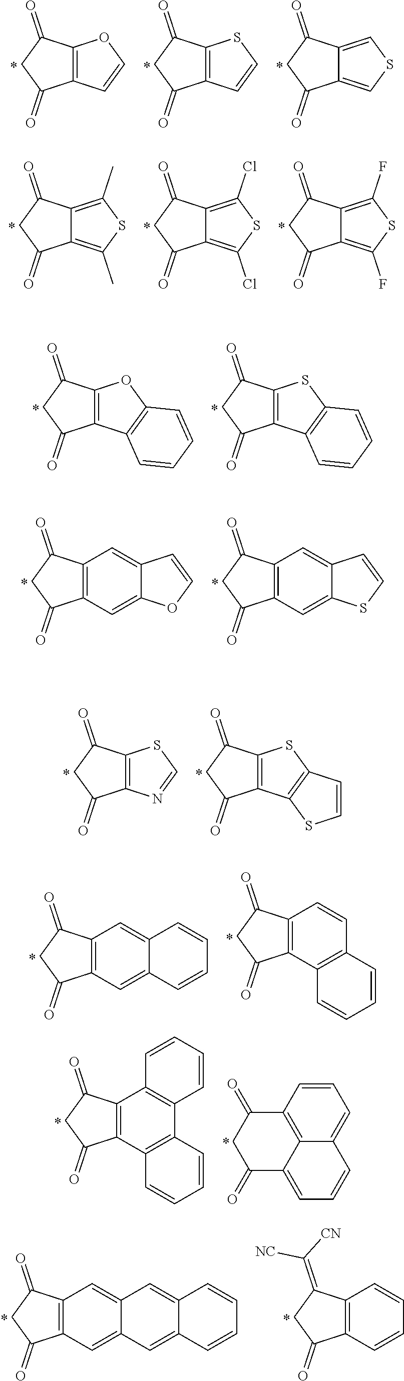

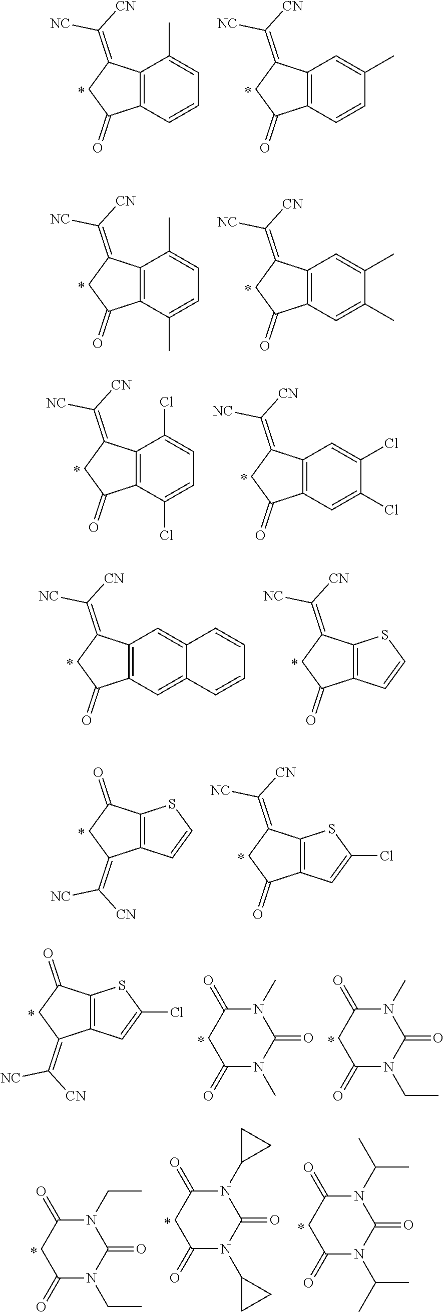

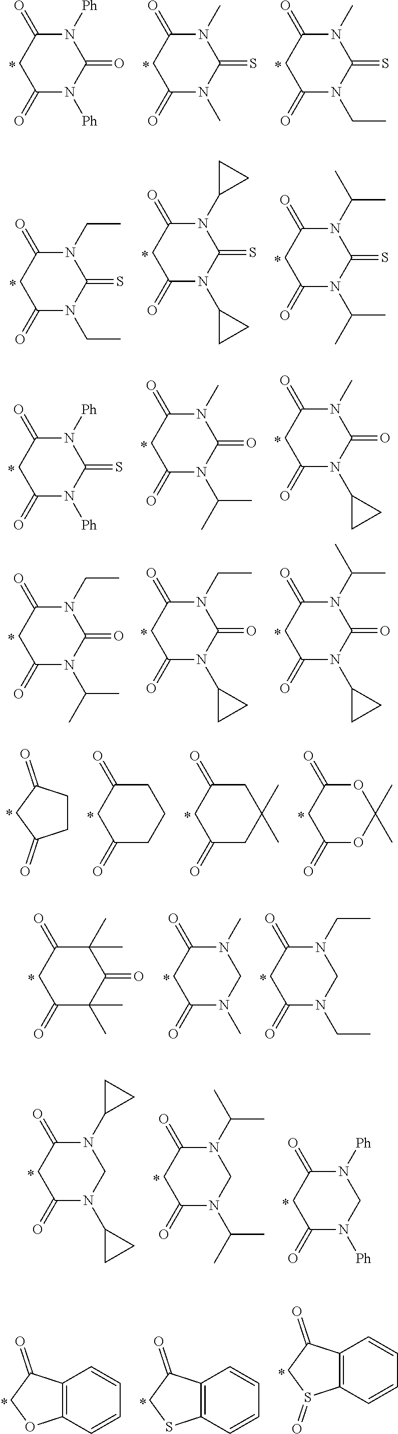

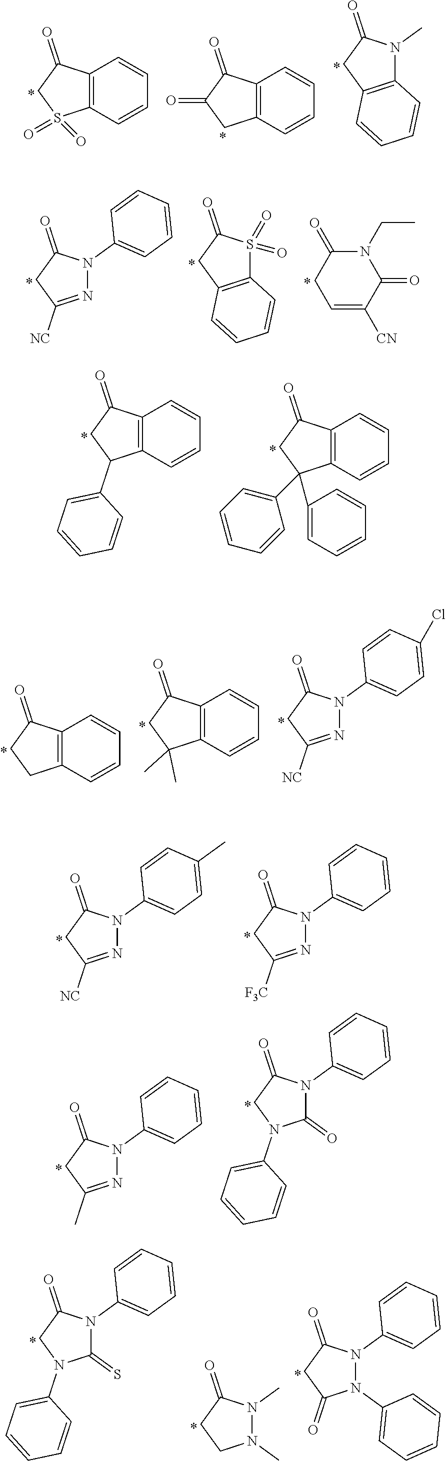

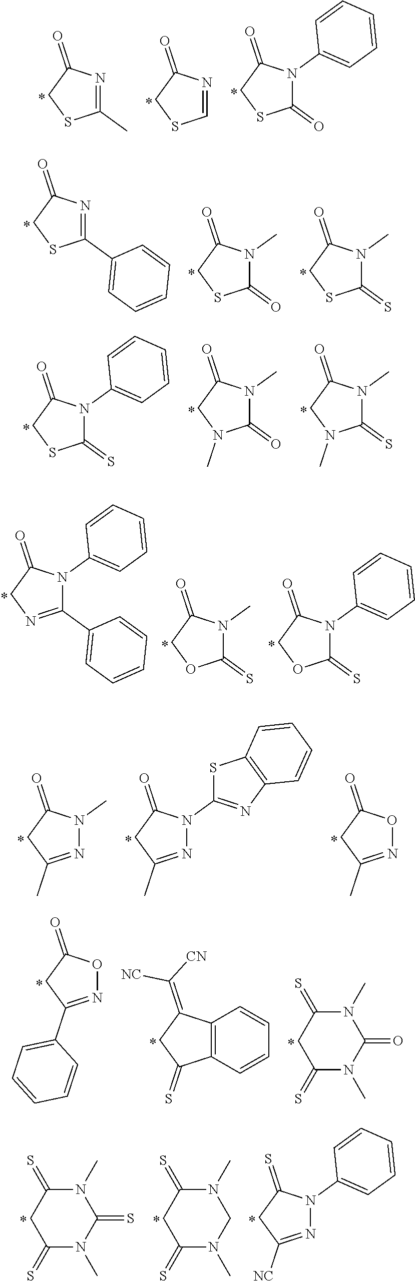

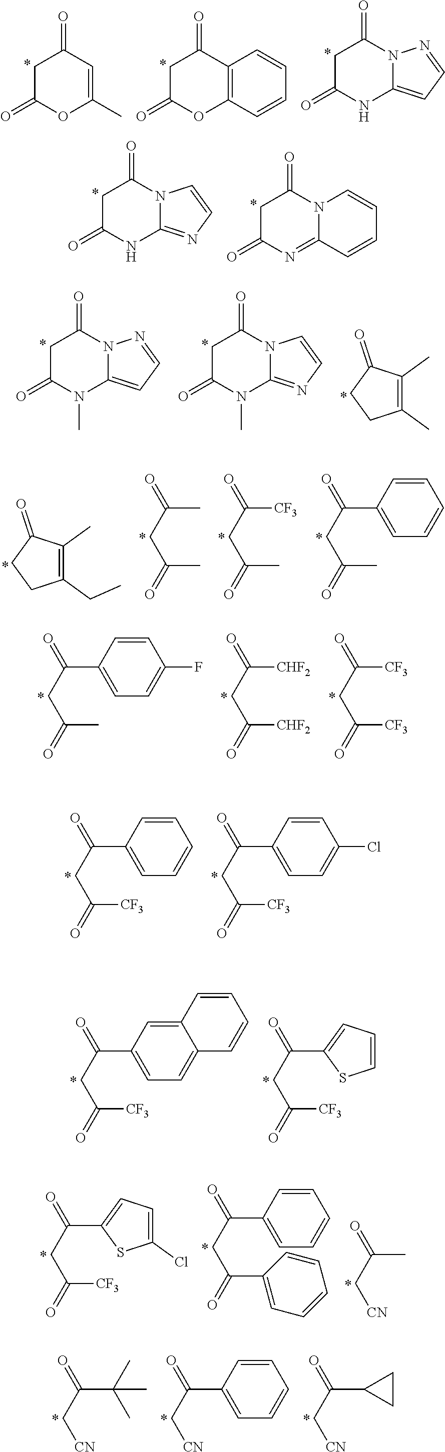

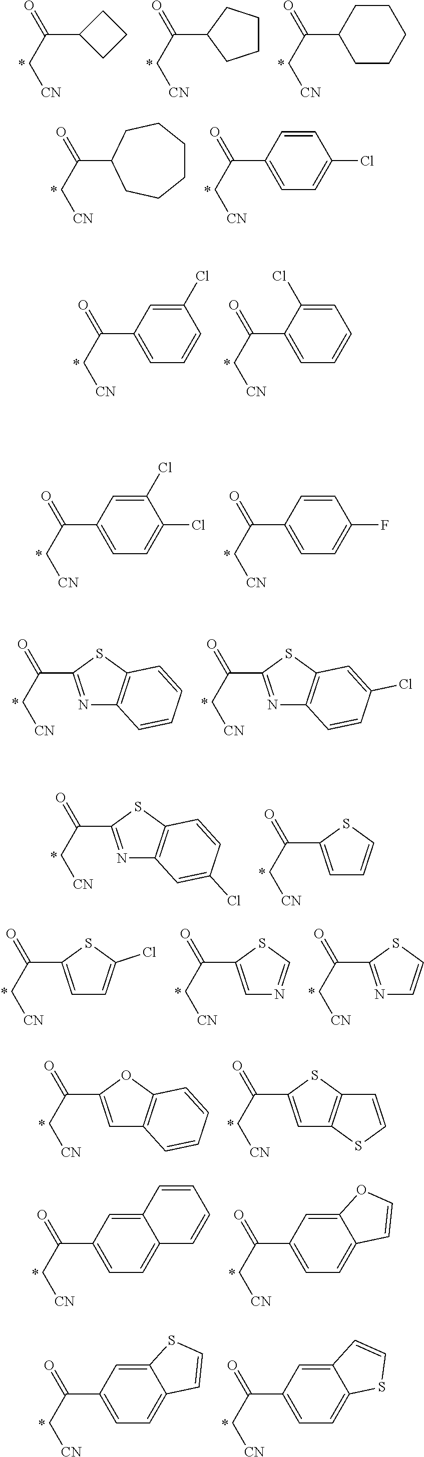

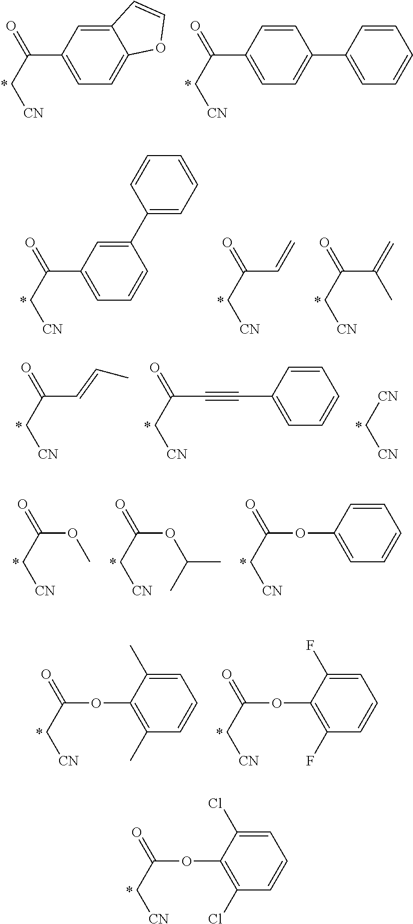

The specific compound may be purified as necessary.

Examples of a purification method of the specific compound include sublimation purification, purification using silica gel column chromatography, purification using gel permeation chromatography, re-slurry washing, re-purification by re-precipitation, purification using an adsorbent such as activated carbon, and recrystallization purification.

A content of the specific compound in the photoelectric conversion film (=Film thickness of specific compound in terms of single layer/Film thickness of photoelectric conversion film×100) is not particularly limited, but is preferably 15% to 75% by volume, more preferably 20% to 60% by volume, and still more preferably 20% to 50% by volume. The specific compound may be used alone or in combination of two or more types thereof. In a case where two or more types thereof are used, it is preferable that the total amount thereof is within the above-described range.

<n-Type Organic Semiconductor>

The photoelectric conversion film preferably contains an n-type organic semiconductor, in addition to the specific compound.

The n-type organic semiconductor is a compound different from the above-described specific compound.

The n-type organic semiconductor is an acceptor-type organic semiconductor material (compound), and refers to an organic compound having a property of easily accepting an electron. That is, the n-type organic semiconductor refers to an organic compound having a larger electron affinity in a case where two organic compounds used in contact with each other. That is, any organic compound having an electron accepting property can be used as the acceptor-type organic semiconductor.

Examples of the n-type organic semiconductor include fullerenes selected from the group consisting of a fullerene and derivatives thereof; fused aromatic carbocyclic compounds (for example, a naphthalene derivative, an anthracene derivative, a phenanthrene derivative, a tetracene derivative, a pyrene derivative, a perylene derivative, a fluoranthene derivative, and the like); heterocyclic compounds with a 5- to 7-membered ring having at least one selected from the group consisting of a nitrogen atom, an oxygen atom, and a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, thiazole, and the like); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivatives and oxadiazole derivatives; anthraquinodimethane derivatives; diphenylquinone derivatives; bathocuproine, bathophenanthroline, and derivatives thereof; triazole compounds; distyrylarylene derivatives; metal complexes having a nitrogen-containing heterocyclic compound as a ligand; silole compounds; 3,4,9,10-perylenetetracarboxylic dianhydride; 3,4,9,10-perylenetetracarboxylic diimide derivatives; and compounds described in paragraphs [0056] and [0057] of JP2006-100767A.

The n-type organic semiconductor (compound) is preferably fullerenes selected from the group consisting of a fullerene and derivatives thereof.

Examples of the fullerene include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the derivatives of fullerene include compounds in which a substituent is added to the above-described fullerenes. The above-described substituent is preferably an alkyl group, an aryl group, or a heterocyclic group. As the derivatives of fullerene, compounds described in JP2007-123707A are preferable.

The n-type organic semiconductor may be an organic coloring agent.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, and a metal complex coloring agent.

A molecular weight of the n-type organic semiconductor is preferably 200 to 1,200 and more preferably 200 to 900.

A maximal absorption wavelength of the n-type organic semiconductor is preferably in a wavelength of 400 nm or less or in a wavelength range of 500 to 600 nm.

It is preferable that the photoelectric conversion film has a bulk heterojunction structure formed in a state in which the specific compound and the n-type organic semiconductor are mixed with each other. The bulk heterojunction structure refers to a layer in which the specific compound and the n-type organic semiconductor are mixed and dispersed in the photoelectric conversion film. The photoelectric conversion film having the bulk heterojunction structure can be formed by a wet method or a dry method. The bulk heterojunction structure is described in detail in paragraphs [0013] and [0014] of JP2005-303266A.

A difference in electron affinity between the specific compound and the n-type organic semiconductor is preferably 0.1 eV or more.

The n-type organic semiconductor may be used alone or in combination of two or more types thereof.

In a case where the photoelectric conversion film contains the n-type organic semiconductor, a content of the n-type organic semiconductor in the photoelectric conversion film (Film thickness of n-type organic semiconductor in terms of single layer/Film thickness of photoelectric conversion film×100) is preferably 15% to 75% by volume, more preferably 20% to 60% by volume, and still more preferably 20% to 50% by volume.

In a case where the n-type organic semiconductor includes fullerenes, a content of the fullerenes to the total content of the n-type organic semiconductor (Film thickness of fullerenes in terms of single layer/Total film thickness of n-type organic semiconductors in terms of single layer×100) is preferably 50% to 100% by volume, and more preferably 80% to 100% by volume. The fullerenes may be used alone or in combination of two or more types thereof.

From the viewpoint of response speed of the photoelectric conversion element, the content of the specific compound to the total content of the specific compound and the n-type organic semiconductor (Film thickness of specific compound in terms of single layer/(Film thickness of specific compound in terms of single layer+Film thickness of n-type organic semiconductor in terms of single layer)×100) is preferably 20% to 80% by volume, and more preferably 40% to 80% by volume.

In a case where the photoelectric conversion film contains the n-type organic semiconductor and a p-type organic semiconductor, the content of the specific compound (Film thickness of specific compound in terms of single layer/(Film thickness of specific compound in terms of single layer+Film thickness of n-type organic semiconductor in terms of single layer+Film thickness of p-type organic semiconductor in terms of single layer)×100) is preferably 15% to 75% by volume, and more preferably 30% to 75% by volume. It is preferable that the photoelectric conversion film substantially contains the specific compound, the n-type organic semiconductor, and the p-type organic semiconductor contained as desired. The term “substantially” indicates that the total content of the specific compound, the n-type organic semiconductor, and the p-type organic semiconductor is 90% to 100% by mass, preferably 95% to 100% by mass, and more preferably 99% to 100% by mass with respect to the total mass of the photoelectric conversion film.

<p-Type Organic Semiconductor>

The photoelectric conversion film preferably contains a p-type organic semiconductor in addition to the above-described specific compound.

The p-type organic semiconductor is a compound different from the above-described specific compound.

The p-type organic semiconductor is a donor-type organic semiconductor material (compound), and refers to an organic compound having a property of easily donating an electron. That is, the p-type organic semiconductor refers to an organic compound having a smaller ionization potential in a case where two organic compounds are used in contact with each other.

The p-type organic semiconductor may be used alone or in combination of two or more types thereof.

Examples of the p-type organic semiconductor include triarylamine compounds (for example, N,N′-diphenyl-N,N′-bis(3-methylphenyl)-(1,1′-biphenyl)-4,4′-diamine (TPD), 4,4′-bis[N-(naphthyl)-N-Phenyl-amino]biphenyl (α-NPD), compounds described in paragraphs [0128] to [0148] of JP2011-228614A, compounds described in paragraphs [0052] to [0063] of JP2011-176259A, compounds described in paragraphs [0119] to [0158] of JP2011-225544A, compounds described in paragraphs [0044] to [0051] of JP2015-153910A, compounds described in paragraphs [0086] to [0090] of JP2012-094660A, and the like), pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1]benzothieno[3,2-b][1]thiophene (BTBT) derivative, a thieno[3,2-f:4,5-f′]bis[1]benzothiophene (TBBT) derivative, compounds described in paragraphs [0031] to [0036] of JP2018-014474A, compounds described in paragraphs [0043] to [0045] of WO2016/194630A, compounds described in paragraphs [0025] to [0037], and [0099] to [0109] of WO2017/159684A, compounds described in paragraphs [0029] to [0034] of JP2017-076766A, compounds described in paragraphs [0015] to [0025] of WO2018/207722A, compounds described in paragraphs [0045] to [0053] of JP2019-054228A, compounds described in paragraphs [0045] to [0055] of WO2019/058995A, compounds described in paragraphs [0063] to [0089] of WO2019/081416A, compounds described in paragraphs [0033] to [0036] of JP2019-080052A, compounds described in paragraphs [0044] to [0054] of WO2019/054125A, compounds described in paragraphs [0041] to [0046] of WO2019/093188A, compounds described in paragraphs [0034] to [0037] of JP2019-050398A, compounds described in paragraphs [0033] to [0036] of JP2018-206878A, compounds described n paragraph [0038] of JP2018-190755A, compounds described in paragraphs [0019] to [0021] of JP2018-026559A, compounds described in paragraphs [0031] to [0056] of JP2018-170487A, compounds described in paragraphs [0036] to [0041] of JP2018-078270A, compounds described in paragraphs [0055] to [0082] of JP2018-166200A, compounds described in paragraphs [0041] to [0050] of JP2018-113425A, compounds described in paragraphs [0044] to [0048] of JP2018-085430A, compounds described in paragraphs [0041] to [0045] of JP2018-056546A, compounds described in paragraphs [0042] to [0049] of JP2018-046267A, compounds described in paragraphs [0031] to [0036] of JP2018-014474A, compounds described in paragraphs [0036] to [0046] of WO2018/016465A, compounds described in paragraphs [0045] to [0048] of JP2020-010024A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, a fluoranthene derivative, and the like), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having a nitrogen-containing heterocyclic compound as a ligand.

As the p-type organic semiconductor, in addition to the above-described compounds, compounds described in JP2021-163968A, JP2022-027575A, JP2022-123944A, JP2022-122839A, JP2022-120323A, JP2022-120273A, JP2022-115832A, JP2022-108268A, JP2022-100258A, JP2022-181226A, and JP2023-005703A can also be used, and the compounds are incorporated in the present specification.

Examples of the p-type organic semiconductor also include compounds having an ionization potential smaller than that of the n-type organic semiconductor, and in a case where this condition is satisfied, the organic coloring agent exemplified as the n-type organic semiconductor can be used.

Compounds which can be used as the p-type organic semiconductor compound are exemplified below.

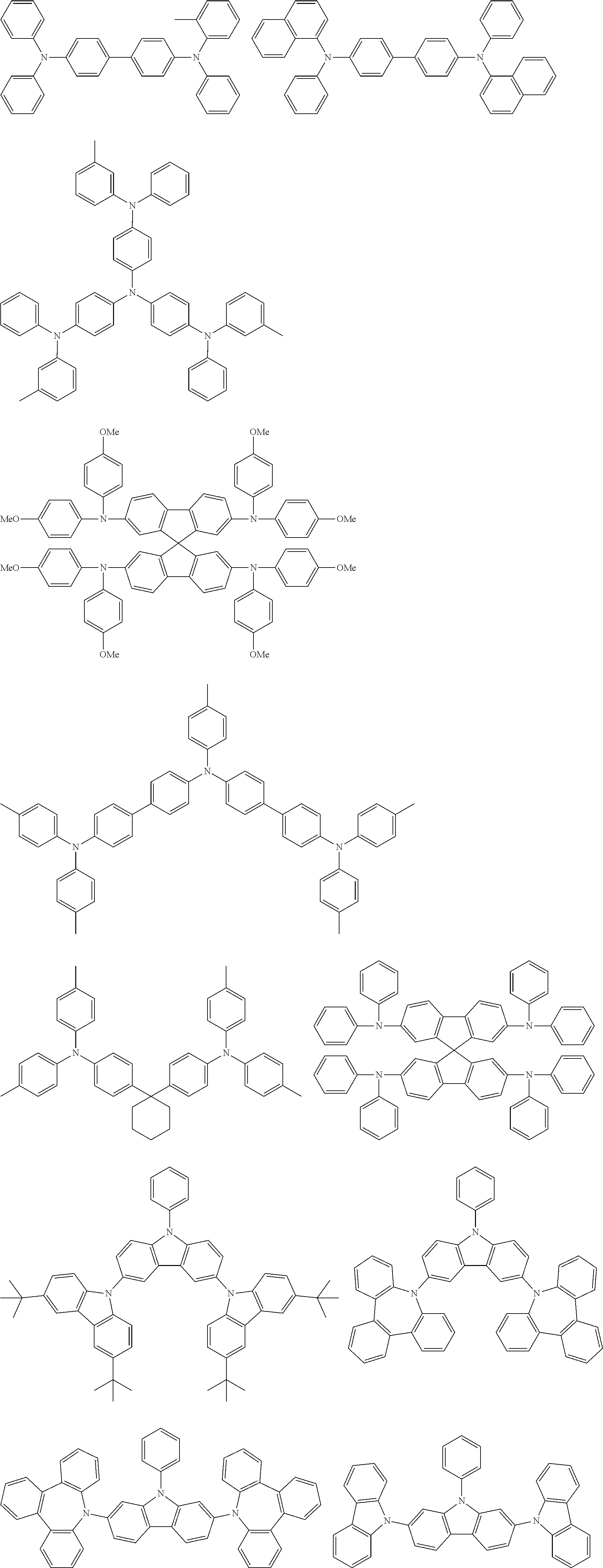

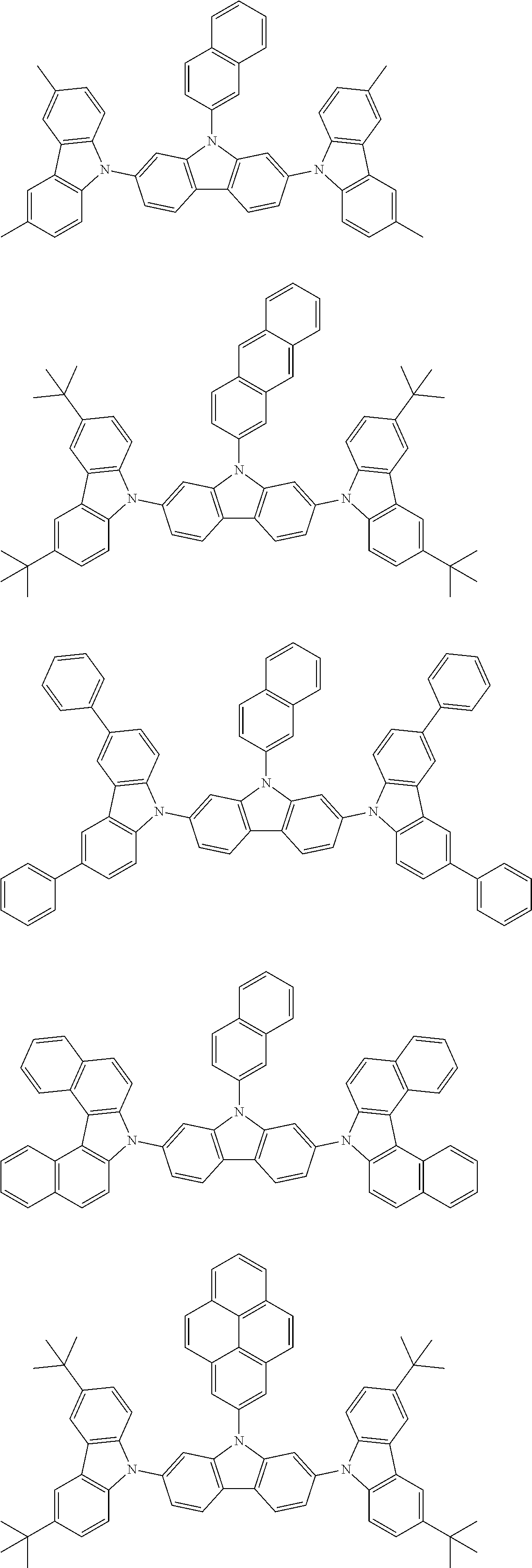

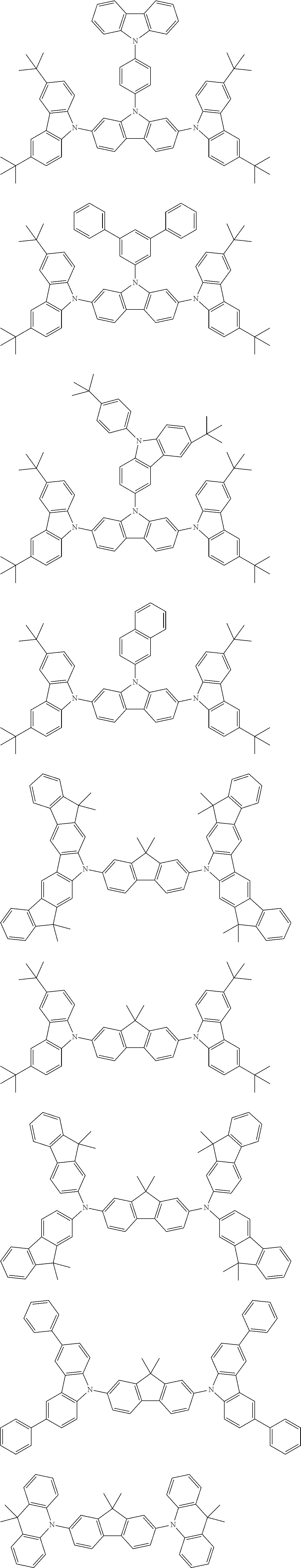

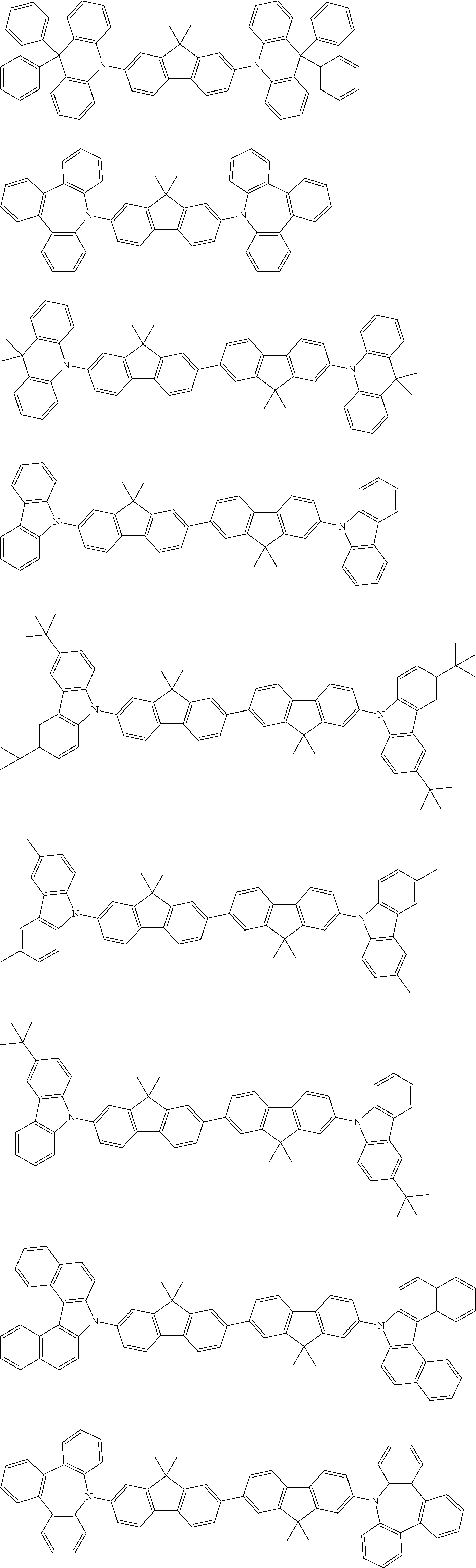

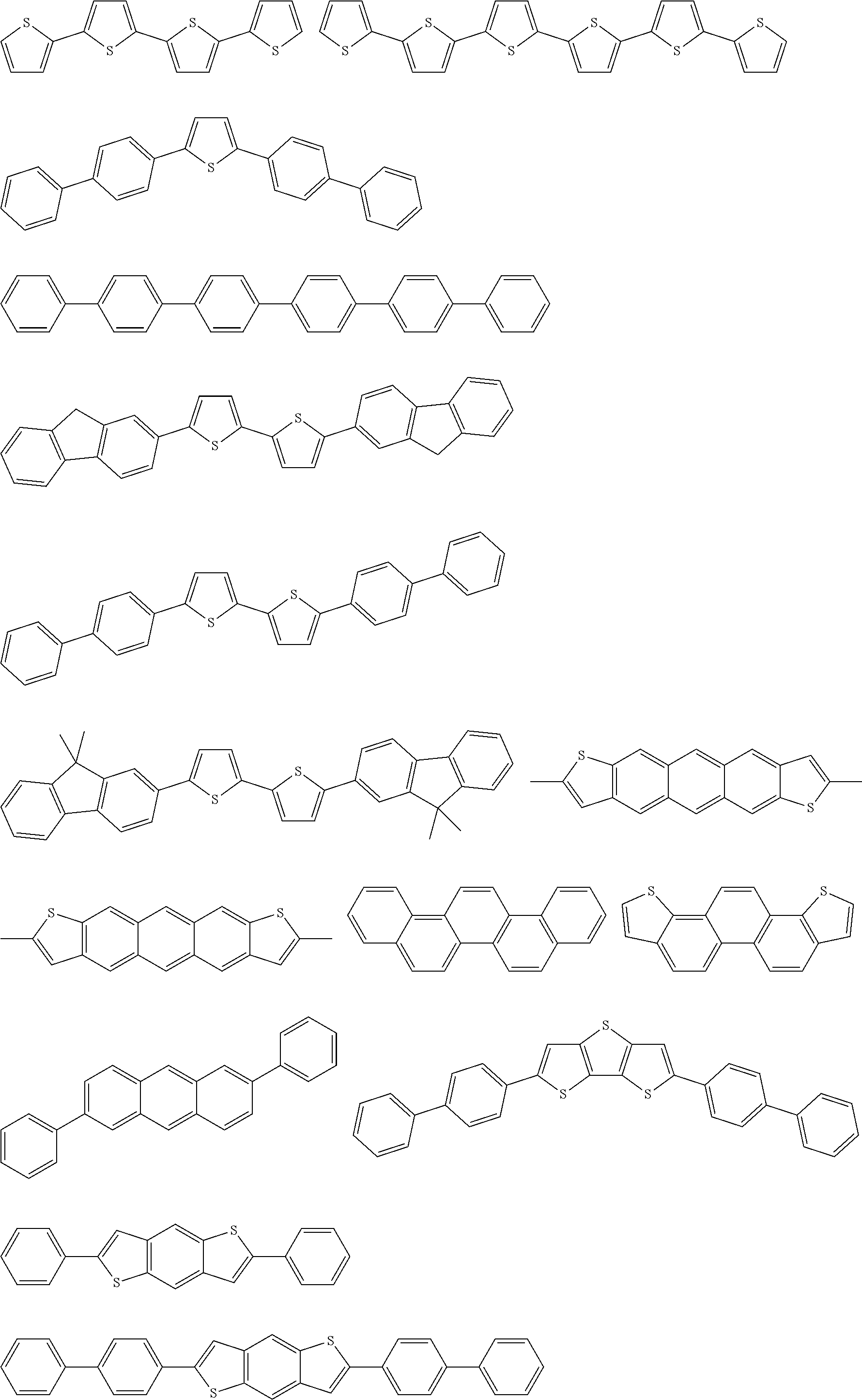

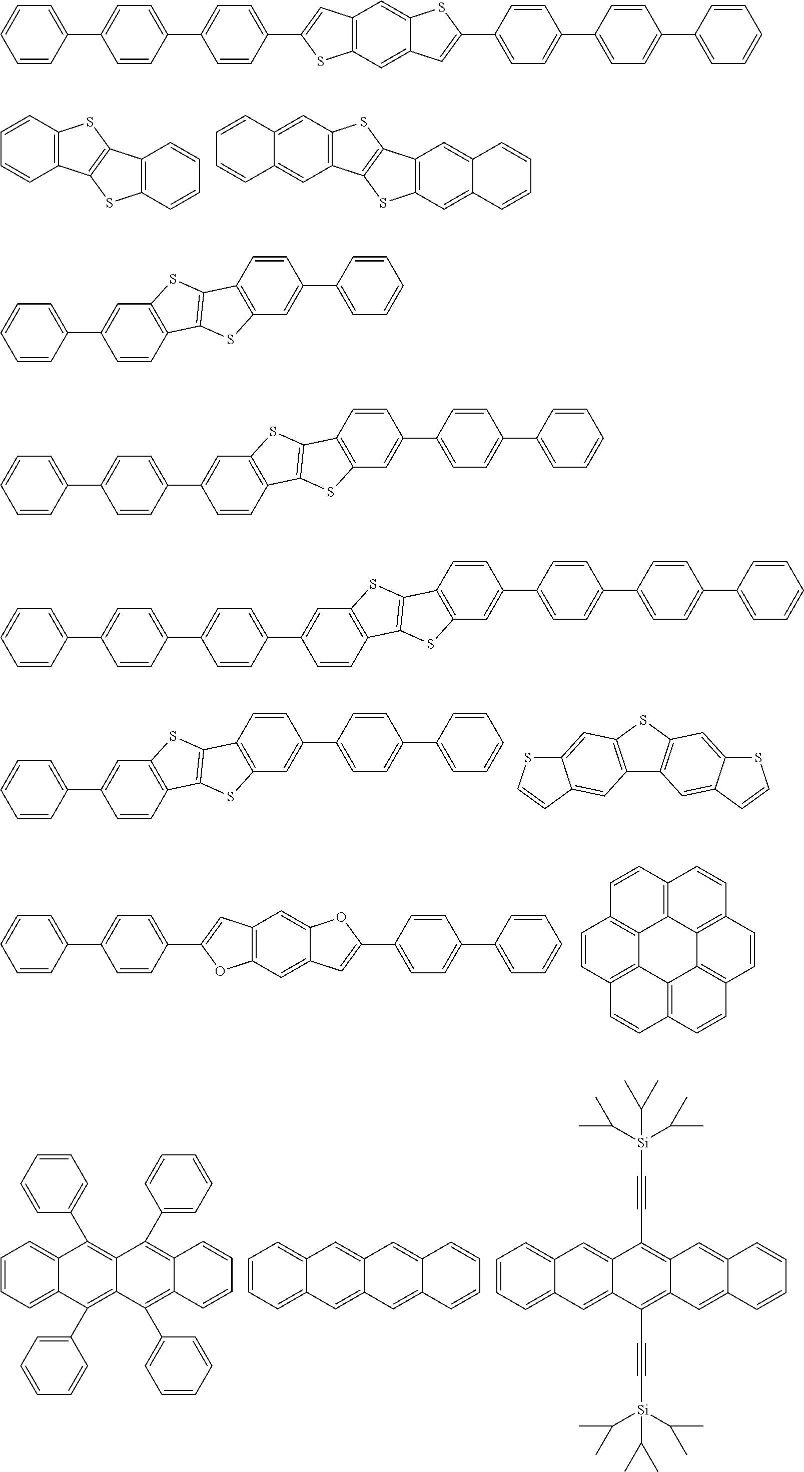

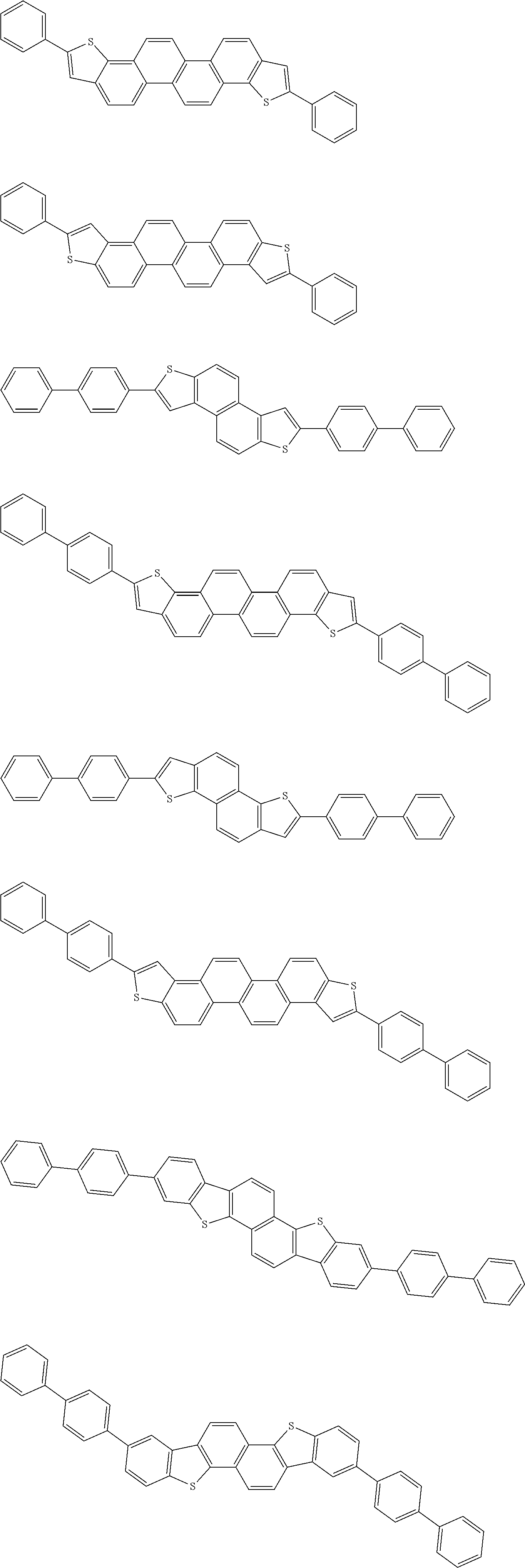

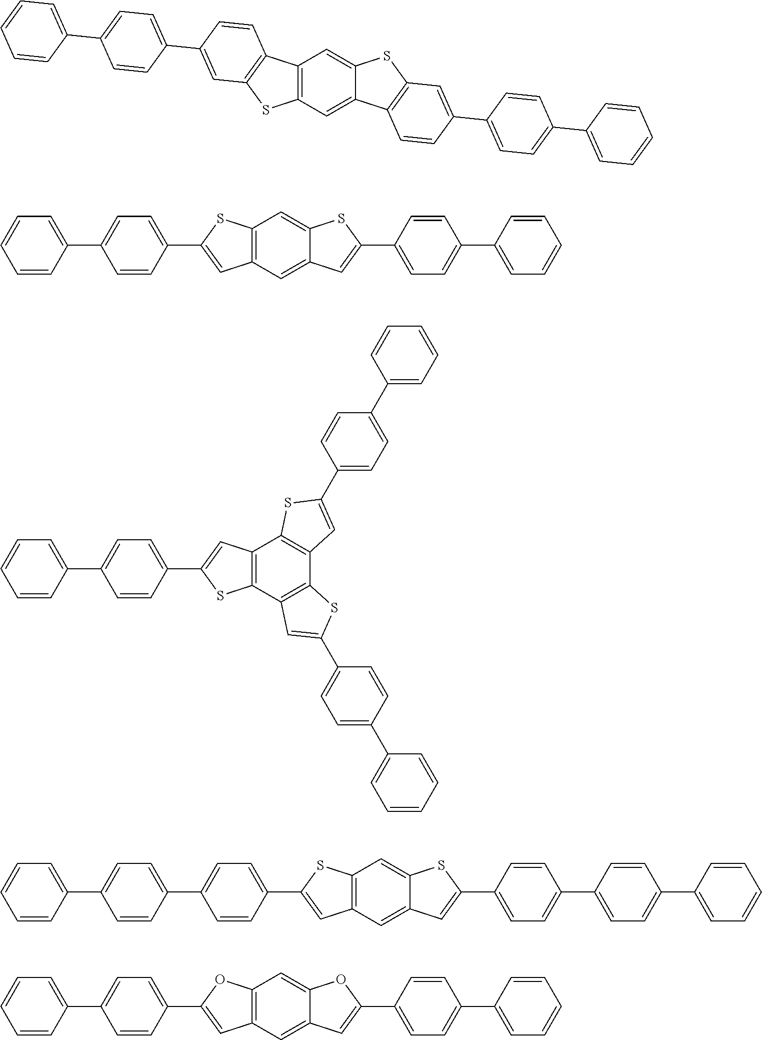 [SMILES: c1ccc(-c2ccc(-c3ccc4c(c3)sc3cc5c(cc34)sc3cc(-c4ccc(-c6ccccc6)cc4)ccc35)cc2)cc1 ; c1ccc(-c2ccc(-c3cc4cc5cc(-c6ccc(-c7ccccc7)cc6)sc5cc4s3)cc2)cc1 ; c1ccc(-c2ccc(-c3cc4c(s3)c3cc(-c5ccc(-c6ccccc6)cc5)sc3c3cc(-c5ccc(-c6ccccc6)cc5)sc43)cc2)cc1 ; c1ccc(-c2ccc(-c3ccc(-c4cc5cc6cc(-c7ccc(-c8ccc(-c9ccccc9)cc8)cc7)sc6cc5s4)cc3)cc2)cc1 ; c1ccc(-c2ccc(-c3cc4cc5cc(-c6ccc(-c7ccccc7)cc6)oc5cc4o3)cc2)cc1]

A difference in ionization potential between the specific compound and the p-type organic semiconductor is preferably 0.1 eV or more.

The p-type organic semiconductor material may be used alone or in combination of two or more types thereof.

In a case where the photoelectric conversion film contains the p-type organic semiconductor, a content of the p-type organic semiconductor in the photoelectric conversion film (Film thickness of p-type organic semiconductor in terms of single layer/Film thickness of photoelectric conversion film×100) is preferably 15% to 75% by volume, more preferably 20% to 60% by volume, and still more preferably 25% to 50% by volume.

The photoelectric conversion film containing the specific compound is a non-luminescent film, and has a feature different from an organic light emitting diode (OLED). The non-luminescent film refers to a film having a light emission quantum efficiency of 1% or less, and the light emission quantum efficiency is preferably 0.5% or less and more preferably 0.1% or less. The lower limit thereof is often 0% or more.

### <Coloring Agent>

The photoelectric conversion film may contain a coloring agent in addition to the above-described specific compound.

The coloring agent is a compound different from the above-described specific compound.

As the coloring agent, an organic coloring agent is preferable.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, and a metal complex coloring agent.

A maximal absorption wavelength of the coloring agent is preferably in the visible light region, more preferably in a wavelength range of 400 to 650 nm, and still more preferably in a wavelength range of 450 to 650 nm.

The coloring agent may be used alone or in combination of two or more types thereof.

A content of the coloring agent with respect to the total content of the specific compound and the coloring agent in the photoelectric conversion film (=(Film thickness of coloring agent in terms of single layer/(Film thickness of specific compound in terms of single layer+Film thickness of coloring agent in terms of single layer)×100)) is preferably 15% to 75% by volume, more preferably 20% to 60% by volume, and still more preferably 20% to 50% by volume.

### <Film Formation Method>

Examples of a film formation method of the above-described photoelectric conversion film include a dry film formation method.

Examples of the dry film formation method include a physical vapor deposition method such as a vapor deposition method (particularly, a vacuum vapor deposition method), a sputtering method, an ion plating method, and a molecular beam epitaxy (MBE) method, and a chemical vapor deposition (CVD) method such as plasma polymerization; and a vacuum vapor deposition method is preferable. In a case where the photoelectric conversion film is formed by the vacuum vapor deposition method, manufacturing conditions such as a degree of vacuum and a vapor deposition temperature can be set according to the conventional method.

A film thickness of the photoelectric conversion film is preferably 10 to 1,000 nm, more preferably 50 to 800 nm, and still more preferably 50 to 500 nm.

### [Electrode]

The photoelectric conversion element preferably includes an electrode.

The electrode (the upper electrode (transparent conductive film) 15 and the lower electrode (conductive film) 11) contains a conductive material. Examples of the conductive material include metals, alloys, metal oxides, electrically conductive compounds, and mixtures thereof.

Since light is incident through the upper electrode 15, the upper electrode 15 is preferably transparent to light to be detected. Examples of a material constituting the upper electrode 15 include conductive metal oxides such as tin oxide doped with antimony, fluorine, or the like (antimony tin oxide (ATO) and fluorine doped tin oxide (FTO)), tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole; and nano carbon materials such as carbon nanotubes and graphene. From the viewpoint of high conductivity and transparency, conductive metal oxides are preferable.

In general, in a case where the conductive film is thinner than a certain range, a resistance value rapidly increases in many cases. In a solid-state imaging element in which the photoelectric conversion element according to the present embodiment is incorporated, a sheet resistance may be 100 to 10,000Ω/□, and a degree of freedom of the film thickness range which can be reduced is large.

In addition, as the film thickness of the upper electrode (transparent conductive film) 15 is thinner, the amount of light which is absorbed in the upper electrode is smaller, and thus light transmittance usually increases. The increase in the light transmittance causes an increase in light absorbance in the photoelectric conversion film and an increase in the photoelectric conversion ability, which is preferable. Considering suppression of leakage current, increase in resistance value of the thin film, and increase in transmittance accompanied by the thinning, the thickness of the upper electrode 15 is preferably 5 to 100 nm, and more preferably 5 to 20 nm.

There is a case where the lower electrode 11 has transparency or an opposite case where the lower electrode does not have transparency and reflects light, depending on use. Examples of a material constituting the lower electrode 11 include conductive metal oxides such as tin oxide doped with antimony, fluorine, or the like (ATO and FTO), tin oxide, zinc oxide, indium oxide, indium tin oxide (ITO), and indium zinc oxide (IZO); metals such as gold, silver, chromium, nickel, titanium, tungsten, and aluminum; conductive compounds such as oxides or nitrides of these metals (for example, titanium nitride (TiN)); mixtures or laminates of these metals and conductive metal oxides; organic conductive materials such as polyaniline, polythiophene, and polypyrrole; and carbon materials such as carbon nanotubes and graphene.

A method of forming the electrode can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the electrode material is ITO, examples thereof include an electron beam method, a sputtering method, a resistance thermal vapor deposition method, a chemical reaction method (such as a sol-gel method), and a coating method with a dispersion of indium tin oxide.

### [Charge Blocking Film: Electron Blocking Film and Hole Blocking Film]

It is preferable that the photoelectric conversion element includes one or more interlayers between the conductive film and the transparent conductive film, in addition to the photoelectric conversion film.

Examples of the above-described interlayer include a charge blocking film. In a case where the photoelectric conversion element includes the film, the characteristics (such as the quantum efficiency and the response speed) of the photoelectric conversion element to be obtained are more excellent. Examples of the charge blocking film include an electron blocking film and a hole blocking film.

### [Electron Blocking Film]

The electron blocking film is a donor-type organic semiconductor material (compound), and the above-described p-type organic semiconductor can be used.

In addition, a polymer material can also be used in the electron blocking film.

Specific examples of the polymer material include a polymer such as phenylenevinylene, fluorene, carbazole, indole, pyrene, pyrrole, picoline, thiophene, acetylene, and diacetylene, and derivatives thereof.

The electron blocking film may be configured by a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the quantum efficiency increases. Examples of the inorganic material which can be used in the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### [Hole Blocking Film]

The hole blocking film is an acceptor-type organic semiconductor material (compound), and the above-described n-type organic semiconductor described above can be used.

The hole blocking film may be configured by a plurality of films.

Examples of a method of manufacturing the charge blocking film include a dry film formation method and a wet film formation method. Examples of the dry film formation method include a vapor deposition method and a sputtering method. The vapor deposition method may be a physical vapor deposition (PVD) method or a chemical vapor deposition (CVD) method, and a physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method; and an inkjet method is preferable from the viewpoint of high-precision patterning.

Each film thickness of the charge blocking films (the electron blocking film and the hole blocking film) is preferably 3 to 200 nm, more preferably 5 to 100 nm, and still more preferably 5 to 30 nm.

### [Substrate]

The photoelectric conversion element may further include a substrate.

Examples of the substrate include a semiconductor substrate, a glass substrate, and a plastic substrate.

As a position of the substrate, in general, the conductive film, the photoelectric conversion film, and the transparent conductive film are laminated on the substrate in this order.

[Sealing Layer] The photoelectric conversion element may further include a sealing layer.

The performance of the photoelectric conversion material may deteriorate significantly due to the presence of deterioration factors such as water molecules. The deterioration can be prevented by coating and sealing the entirety of the photoelectric conversion film with a sealing layer such as diamond-like carbon (DLC) and ceramics such as metal oxide, metal nitride, or metal nitride oxide, which are dense and into which water molecules do not permeate.

Examples of the sealing layer include sealing layers described in paragraphs [0210] to [0215] of JP2011-082508A, the contents of which are incorporated herein by reference.

### [Imaging Element]

Examples of the application of the photoelectric conversion element include an imaging element.

The imaging element is an element which converts optical information of an image into the electric signal, and is usually an element in which a plurality of photoelectric conversion elements are arranged in a matrix on the same plane, optical signals are converted into electric signals in each photoelectric conversion element (a pixel), and the electric signals can be sequentially output to the outside of the imaging elements for each pixel. Therefore, each pixel is formed of one or more photoelectric conversion elements and one or more transistors.

### [Optical Sensor]

Examples of another application of the photoelectric conversion element include a photoelectric cell and a optical sensor, but it is preferable that the photoelectric conversion element according to the embodiment of the present invention is used as the optical sensor. The above-described photoelectric conversion element may be used alone as the optical sensor, or may be used as a line sensor in which the photoelectric conversion elements are linearly arranged or as a two-dimensional sensor in which the photoelectric conversion elements are arranged on a plane.

### [Compound]

The present invention also includes a compound. The compound according to the embodiment of the present invention is the above-described specific compound.

### EXAMPLES

Hereinbelow, the present invention will be described in more detail with reference to Examples.

The materials, the amounts of materials used, the proportions, the treatment details, the treatment procedure, and the like shown in Examples below may be modified as appropriate as long as the modifications do not depart from the spirit of the present invention. Therefore, the scope of the present invention should not be construed as being limited to Examples shown below.

### [Materials Used for Producing Photoelectric Conversion Element]

Each material used for producing a photoelectric conversion element is shown below.

### Synthesis of Compound B-3 (Synthesis Example of Specific Compound)

A compound B-3, which is the specific compound in the present invention, was synthesized according to the following scheme. A structure of the compound B-3 was confirmed by LDI-MS.

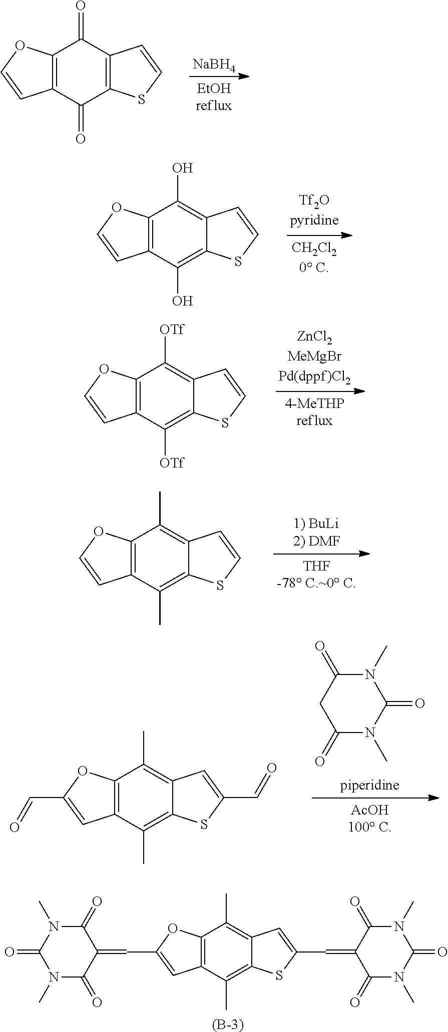

Specific compounds and comparative compounds used in the photoelectric conversion film, other than the compound B-3, were synthesized with reference to [Synthesis of compound B-3] described above.

Each material used for the photoelectric conversion element is shown below. The compounds B-1 to B-23 correspond to the specific compounds used in Examples; and the compounds C1 and C2 correspond to the comparative compounds used in Comparative Examples.

### [Specific Compound and Comparative Compound]

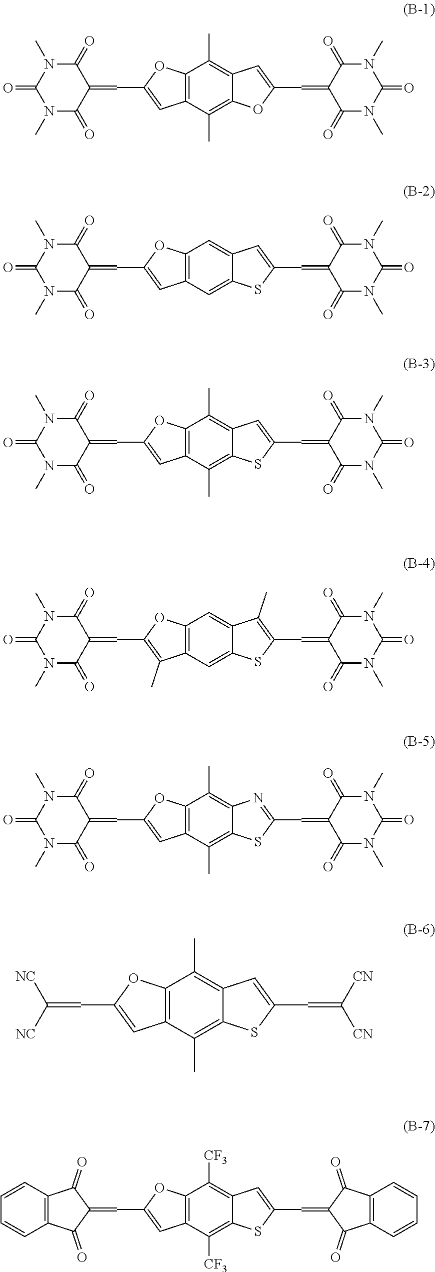

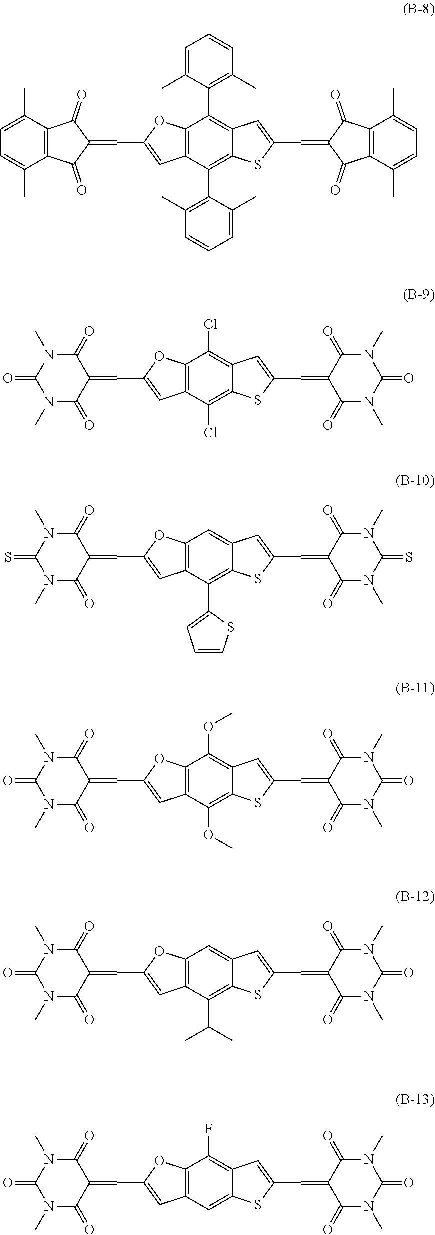

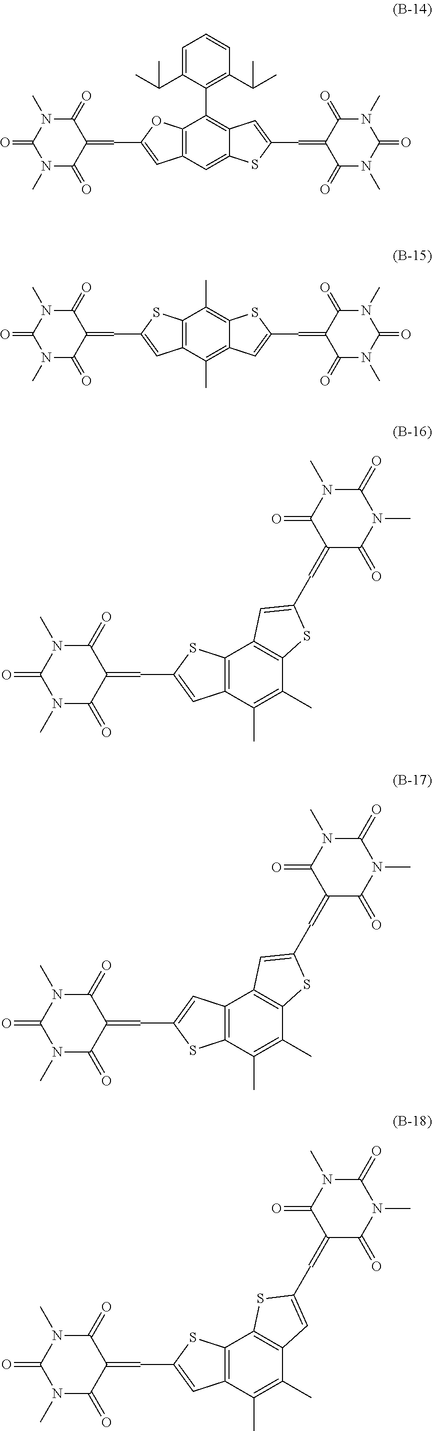 [SMILES: CC(C)c1cccc(C(C)C)c1-c1c2cc(C=C3C(=O)N(C)C(=O)N(C)C3=O)sc2cc2cc(C=C3C(=O)N(C)C(=O)N(C)C3=O)oc12 ; Cc1c2cc(C=C3C(=O)N(C)C(=O)N(C)C3=O)sc2c(C)c2sc(C=C3C(=O)N(C)C(=O)N(C)C3=O)cc12 ; Cc1c(C)c2sc(C=C3C(=O)N(C)C(=O)N(C)C3=O)cc2c2sc(C=C3C(=O)N(C)C(=O)N(C)C3=O)cc12 ; Cc1c(C)c2sc(C=C3C(=O)N(C)C(=O)N(C)C3=O)cc2c2cc(C=C3C(=O)N(C)C(=O)N(C)C3=O)sc12 ; Cc1c(C)c2cc(C=C3C(=O)N(C)C(=O)N(C)C3=O)sc2c2sc(C=C3C(=O)N(C)C(=O)N(C)C3=O)cc12]

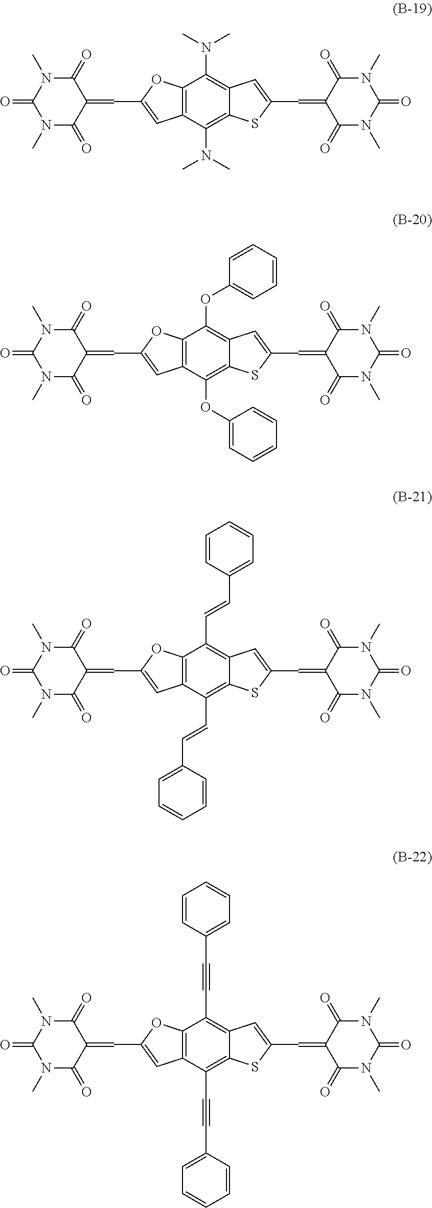 [SMILES: CN1C(=O)C(=Cc2cc3c(N(C)C)c4sc(C=C5C(=O)N(C)C(=O)N(C)C5=O)cc4c(N(C)C)c3o2)C(=O)N(C)C1=O ; CN1C(=O)C(=Cc2cc3c(Oc4ccccc4)c4sc(C=C5C(=O)N(C)C(=O)N(C)C5=O)cc4c(Oc4ccccc4)c3o2)C(=O)N(C)C1=O ; CN1C(=O)C(=Cc2cc3c(/C=C/c4ccccc4)c4sc(C=C5C(=O)N(C)C(=O)N(C)C5=O)cc4c(/C=C/c4ccccc4)c3o2)C(=O)N(C)C1=O ; CN1C(=O)C(=Cc2cc3c(C#Cc4ccccc4)c4sc(C=C5C(=O)N(C)C(=O)N(C)C5=O)cc4c(C#Cc4ccccc4)c3o2)C(=O)N(C)C1=O]

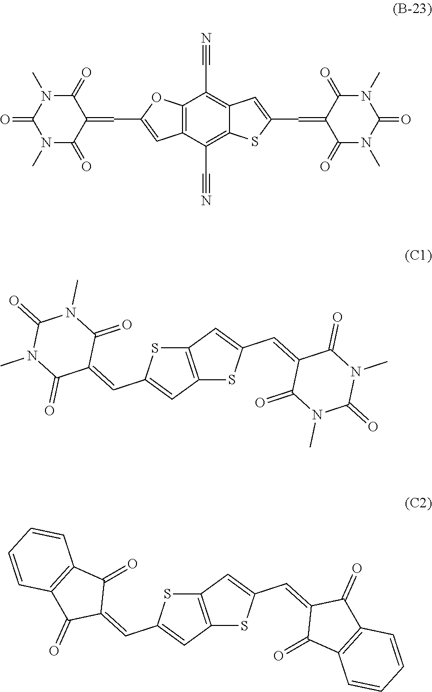

[n-Type Organic Semiconductor]
C₆₀: fullerene (C₆₀)
   [p-Type Organic Semiconductor]

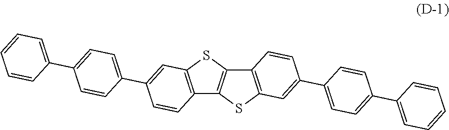 [SMILES: c1ccc(-c2ccc(-c3ccc4c(c3)sc3c5ccc(-c6ccc(-c7ccccc7)cc6)cc5sc43)cc2)cc1]

### [Coloring Agent]

In Test Y described later, the following coloring agents were used.

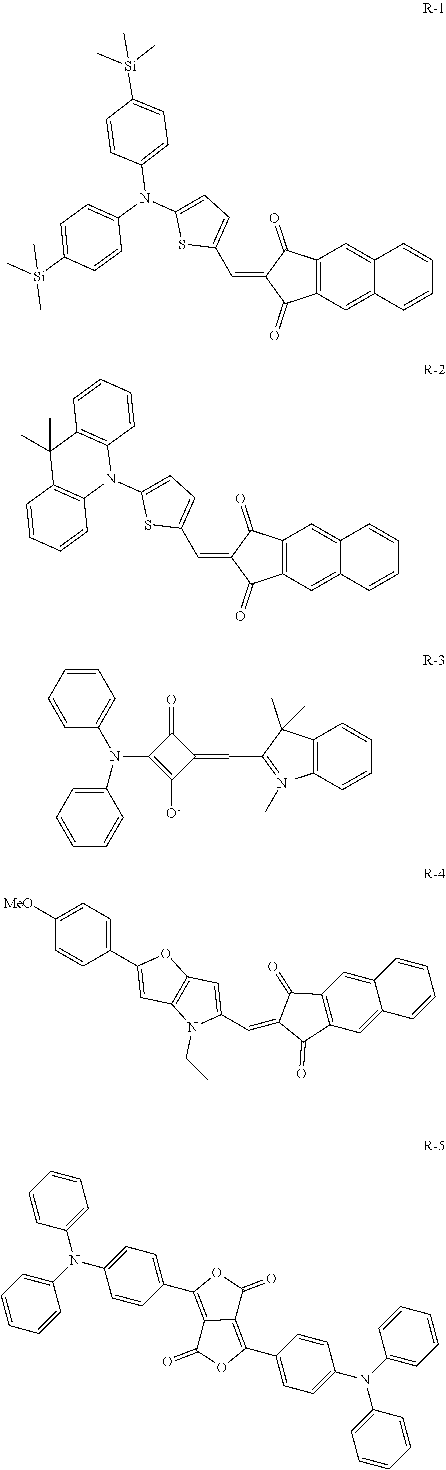 [SMILES: C[Si](C)(C)c1ccc(N(c2ccc([Si](C)(C)C)cc2)c2ccc(C=C3C(=O)c4cc5ccccc5cc4C3=O)s2)cc1 ; CC1(C)c2ccccc2N(c2ccc(C=C3C(=O)c4cc5ccccc5cc4C3=O)s2)c2ccccc21 ; C[N+]1=C(C=C2C(=O)C(N(c3ccccc3)c3ccccc3)=C2[O-])C(C)(C)c2ccccc21 ; CCn1c(C=C2C(=O)c3cc4ccccc4cc3C2=O)cc2oc(-c3ccc(OC)cc3)cc21 ; O=C1OC(c2ccc(N(c3ccccc3)c3ccccc3)cc2)=C2C(=O)OC(c3ccc(N(c4ccccc4)c4ccccc4)cc3)=C12]

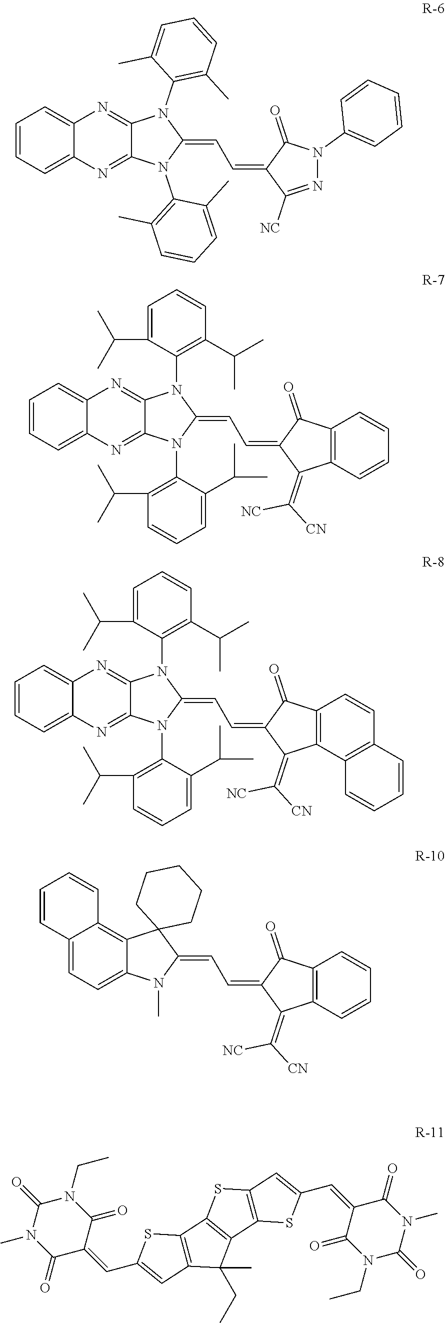

A photoelectric conversion element was produced using the above-described materials, and Test X and Test Y were performed.

### [Test X]

### <Production of Photoelectric Conversion Element>

A photoelectric conversion element having the form of FIG. 2 was produced using the various components shown above. Here, the photoelectric conversion element included a lower electrode 11, an electron blocking film 16A, a photoelectric conversion film 12, a hole blocking film 16B, and an upper electrode 15.

Specifically, an amorphous ITO was formed into a film on a glass substrate by a sputtering method to form the lower electrode 11 (thickness: 30 nm), and the compound (EB-1) was further formed into a film on the lower electrode 11 by a vacuum thermal vapor deposition method to form the electron blocking film 16A (thickness: 30 nm).

Subsequently, the glass substrate was subjected to a vacuum vapor deposition method in a state of room temperature to form a film by co-vapor deposition of each specific compound or each comparative compound shown in Table 1 on the electron blocking film 16A, and the n-type organic semiconductor (fullerene (C₆₀)) and the p-type organic semiconductor (D-1) were each subjected to a co-vapor deposition method so that the thickness thereof was 90 nm in terms of single layer. As a result, the photoelectric conversion film 12 having a bulk heterojunction structure with 270 nm was formed. In this case, a film formation rate of the photoelectric conversion film 12 was set to 1.0 Å/sec.

Furthermore, the compound (EB-2) was vapor-deposited on the photoelectric conversion film 12 to form the hole blocking film 16B (thickness: 10 nm). Amorphous ITO was formed into a film on the hole blocking film 16B by a sputtering method to form the upper electrode 15 (transparent conductive film) (thickness: 10 nm). An SiO film was formed as a sealing layer on the upper electrode 15 by a vacuum vapor deposition method, an aluminum oxide (Al₂O₃) layer was formed thereon by an atomic layer chemical vapor deposition (ALCVD) method, and the obtained laminate was heated in a glove box at 150° C. for 30 minutes to obtain a photoelectric conversion element of each of Examples and Comparative Examples.

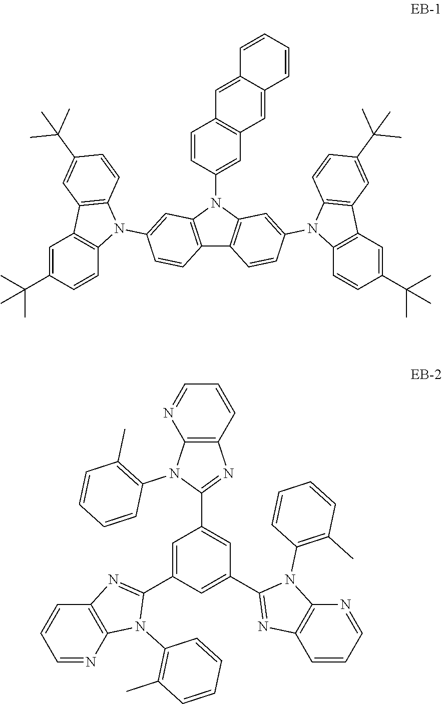 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1 ; Cc1ccccc1-n1c(-c2cc(-c3nc4cccnc4n3-c3ccccc3C)cc(-c3nc4cccnc4n3-c3ccccc3C)c2)nc2cccnc21]

<Dark current>

A dark current of each obtained photoelectric conversion element was measured by the following method.

A voltage was applied to the lower electrode and the upper electrode of each of the photoelectric conversion elements with an electric field strength of 2.5×10¹ V/cm, and a current value (dark current) in a dark place was measured. As a result, it was confirmed that all of the photoelectric conversion elements had a dark current of 50 nA/cm² or less, which indicates that all of the photoelectric conversion elements had a sufficiently low dark current.

### <Quantum Efficiency (Sensitivity)>

For the photoelectric conversion element of each of Examples and each of Comparative Examples, the quantum efficiency (sensitivity) with respect to blue and green light was measured by the following method.

A voltage was applied to the photoelectric conversion element with an electric field strength of 2.0×10′ V/cm, and then light was emitted from the upper electrode (transparent conductive film) side to measure quantum efficiency (photoelectric conversion efficiency) at a wavelength of 460 nm. The quantum efficiency (relative ratio) was calculated according to Expression (S1). From the obtained values, the quantum efficiency was evaluated according to the following evaluation standard.

Expression (S1): Quantum efficiency (relative ratio)=(Photoelectric conversion efficiency of each of Examples or each of Comparative Examples)/(Photoelectric conversion efficiency of Example 1-6)

(Evaluation standard)
A: quantum efficiency (relative ratio) was 1.1 or more.
B: quantum efficiency (relative ratio) was 1.0 or more and less than 1.1.
C: quantum efficiency (relative ratio) was 0.9 or more and less than 1.0.
D: quantum efficiency (relative ratio) was 0.8 or more and less than 0.9.
E: quantum efficiency (relative ratio) was less than 0.8.

### <Response Speed>

For the photoelectric conversion element of each of Examples and each of Comparative Examples, the response speed with respect to blue and green light was measured by the following method.

A voltage was applied to the photoelectric conversion element with an electric field strength of 2.0×10¹ V/cm. Thereafter, a light emitting diode (LED) was turned on for an instant to emit light from the upper electrode (transparent conductive film) side, a photocurrent at a wavelength of 460 nm was measured with an oscilloscope, a rise time until the signal intensity rose from 0% to 97% was measured, and the relative response speed was calculated according to Expression (S2). From the obtained values, the response speed was evaluated according to the following evaluation standard.

Expression (S2): Relative response speed=(Rise time of each of Examples or each of Comparative Examples)/(Rise time of Example 1-6)

### (Evaluation Standard)

A: relative response speed was 0.5 or more and less than 1.0.
B: relative response speed was 1.0 or more and less than 1.5.
C: relative response speed was 1.5 or more and less than 2.0.
D: relative response speed was 2.0 or more and less than 2.5.
E: relative response speed was 2.5 or more.

### <Electric Field Strength Dependence of Response Speed>

For each obtained photoelectric conversion element, electric field strength dependence of the response speed was evaluated by the following method.

A voltage was applied to the photoelectric conversion element with an electric field strength of 2.0×10′ V/cm. Thereafter, a light emitting diode (LED) was turned on for an instant to emit light from the upper electrode (transparent conductive film) side, a photocurrent at this time at a wavelength of 460 nm was measured with an oscilloscope, and a rise time until the signal intensity rose from 0% to 97% was measured.

Subsequently, a response speed at 7.5×10′ V/cm was measured by the same procedure as in the evaluation of the response speed, except that the voltage applied to each photoelectric conversion element was changed to 7.5×10¹ V/cm.

From the obtained values of the response speed at 2.0×10′ V/cm and the response speed at 7.5×10′ V/cm, the electric field strength dependence of the response speed was determined according to Expression (S1) and evaluated.

Expression (S1): Electric field strength dependence of response speed=(Rise time at 7.5×10′ V/cm at wavelength of 460 nm in each of Examples or Comparative Examples)/(Rise time at 2.0×10′ V/cm at wavelength of 460 nm in each of Examples or Comparative Examples)

In Expression (S1), each photoelectric conversion element used for the numerator and the denominator are the same.

For example, with regard to Example 1-1, the rise time of the photoelectric conversion efficiency at 7.5×10¹ V/cm and a wavelength of 460 nm in Example 1-1 and the rise time of the photoelectric conversion efficiency at 2.0×10⁵ V/cm and a wavelength of 460 nm in Example 1-1 were compared.

### (Evaluation Standard)

AA: electric field strength dependence of the response speed was less than 2.0.
A: electric field strength dependence of the response speed was 2.0 or more and less than 2.6.
B: electric field strength dependence of the response speed was 2.6 or more and less than 3.4.
C: electric field strength dependence of the response speed was 3.4 or more and less than 4.2.
D: electric field strength dependence of the response speed was 4.2 or more and less than 5.0.
E: electric field strength dependence of the response speed was 5.0 or more.

Table 1 shows the above-described evaluation results.

The respective notations in Table 1 indicate the following.

In the column of “Rs”, a case where at least one of Y²¹, . . . , or Y²⁴ in Formula (2) represented —CRs=, at least one of Y³¹, . . . , or Y³⁴ in Formula (3) represented —CRs=, at least one of Y⁴¹, . . . , or Y⁴⁴ in Formula (4) represented —CRs=, at least one of Y⁵¹, . . . , or Y⁵⁴ in Formula (5) represented —CRs=, or at least one of Y⁶¹, . . . , or Y⁶⁴ in Formula (6) represented —CRs=, in which Rs's each independently represented the group selected from the substituent group S, is indicated as “A”, and a case other than the above case is indicated as “B”.

In the column of “Formulae (2) to (6) limitation”, a case where D in Formula (1) represented the group represented by any of Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), or Formula (6-12) is indicated as “A”, and a case other than the above case is indicated as “B”. In the column of “CR^{Y1}”, a case where, in Formulae (2) to (6), Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ each independently represented —CR^{Y1}═ is indicated as “A”, and a case other than the above case is indicated as “B”.

In the column of “Formula (A-1)”, a case where A¹ and A² in Formula (1) each independently represented the group represented by Formula (A-1) is indicated as “A”, and a case other than the above case is indicated as “B”.

In the column of “Rs limitation 1”, in Formula (1), a case where the requirement described in the column of “Formulae (2) to (6) limitation” was satisfied, Rs represented a group selected from the group consisting of an alkyl group which may have a substituent, an aryl group which may have a substituent, a heteroaryl group which may have a substituent, and a halogen atom, and a plurality of Rs's were present and the groups were different from each other is indicated as “A”, and a case other than the above case is indicated as “B”. In the column of “Rs limitation 2”, in Formula (1), a case where the requirement described in the column of “Formulae (2) to (6) limitation” was satisfied, Rs represented a group selected from the group consisting of an alkyl group which may have a substituent and an aryl group which may have a substituent, and a plurality of Rs's were present and the groups were different from each other is indicated as “A”, and a case other than the above case is indicated as “B”.

TABLE 1

| | | | | | | | | Evaluation | | |
|---|---|---|---|---|---|---|---|---|---|---|
| | | | | | | | | | | Electric |
| | | | | | | | | | | field |
| | | | | | | | | | | strength |
| | | | Formulae | | | Rs | Rs | | | dependence of |
| | | | (2) to (6) | | Formula | limitation | limitation | | Response | response |
| | Compound | Rs | limitation | CR^{Y1} | (A-1) | 1 | 2 | Sensitivity | speed | speed |
| Example 1-1 | B-1 | A | A | A | A | B | B | A | A | B |
| Example 1-2 | B-2 | B | B | A | A | B | B | C | C | C |
| Example 1-3 | B-3 | A | A | A | A | B | B | A | A | B |
| Example 1-4 | B-4 | A | B | A | A | B | B | B | B | C |
| Example 1-5 | B-5 | A | A | B | A | B | B | B | B | C |
| Example 1-6 | B-6 | A | A | A | B | B | B | B | B | C |
| Example 1-7 | B-7 | A | A | A | A | B | B | A | A | B |
| Example 1-8 | B-8 | A | A | A | A | B | B | A | A | B |
| Example 1-9 | B-9 | A | A | A | A | B | B | A | A | B |
| Example 1-10 | B-10 | A | A | A | A | A | B | A | A | A |
| Example 1-11 | B-11 | A | A | A | A | B | B | A | A | B |
| Example 1-12 | B-12 | A | A | A | A | A | A | A | A | AA |
| Example 1-13 | B-13 | A | A | A | A | A | B | A | A | A |
| Example 1-14 | B-14 | A | A | A | A | A | A | A | A | AA |
| Example 1-15 | B-15 | A | A | A | A | B | B | A | B | B |
| Example 1-16 | B-16 | A | A | A | A | B | B | A | B | B |
| Example 1-17 | B-17 | A | A | A | A | B | B | A | B | B |
| Example 1-18 | B-18 | A | A | A | A | B | B | A | B | B |
| Example 1-19 | B-19 | A | A | A | A | B | B | A | A | B |
| Example 1-20 | B-20 | A | A | A | A | B | B | A | A | B |
| Example 1-21 | B-21 | A | A | A | A | B | B | A | A | B |
| Example 1-22 | B-22 | A | A | A | A | B | B | A | A | B |
| Example 1-23 | B-23 | A | A | A | A | B | B | A | A | B |
| Comparative | C1 | — | — | — | — | — | — | E | E | E |
| Example 1-1 | | | | | | | | | | |
| Comparative | C2 | — | — | — | — | — | — | E | E | E |
| Example 1-2 | | | | | | | | | | |

As is clear from the results in the table, it was found that the photoelectric conversion elements according to Examples of the present invention exhibited a desired effect.

On the other hand, the photoelectric conversion elements of Comparative Examples, using the comparative compound not corresponding to the specific compound, did not exhibit the above-described effect sufficiently.

In addition, from the comparison of Example 1-2 and Example 1-4, it was found that, in a case where at least one of Y²¹, . . . , or Y²⁴ in Formula (2) represented —CRs=, at least one of Y³¹, or Y³⁴ in Formula (3) represented —CRs=, at least one of Y⁴¹, . . . , or Y⁴⁴ in Formula (4) represented —CRs=, at least one of Y⁵¹, . . . , or Y⁵⁴ in Formula (5) represented —CRs=, or at least one of Y⁶¹, . . . , or Y⁶⁴ in Formula (6) represented —CRs=, in which Rs's each independently represented the group selected from the substituent group S, the sensitivity and the response speed were more excellent.

From the comparison of Example 1-1 and Example 1-4, it was found that, in a case where D in Formula (1) represented the group represented by any of Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), or Formula (6-12), the sensitivity, the response speed, and the electric field strength dependence of the response speed were more excellent.

From the comparison of Example 1-1 and Example 1-5, it was found that, in a case where Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ in Formulae (2) to (6) each independently represented —CR^{Y1}=, the sensitivity, the response speed, and the electric field strength dependence of the response speed were more excellent.

From the comparison of Example 1-1 and Example 1-6, it was found that, in a case where A¹ and A² in Formula (1) each independently represented the group represented by Formula (A-1), the sensitivity, the response speed, and the electric field strength dependence of the response speed were more excellent.

From the comparison of Example 1-1 and Example 1-10, it was found that, in a case where, in Formula (1), the requirement described in the column of “Formulae (2) to (6) limitation” was satisfied, Rs represented a group selected from the group consisting of an alkyl group which may have a substituent, an aryl group which may have a substituent, a heteroaryl group which may have a substituent, and a halogen atom, and a plurality of Rs's were present and the groups were different from each other, the electric field strength dependence of the response speed was more excellent.

From the comparison of Example 1-12 and Example 1-13, it was found that, in a case where, in Formula (1), the requirement described in the column of “Formulae (2) to (6) limitation” was satisfied, Rs represented a group selected from the group consisting of an alkyl group which may have a substituent and an aryl group which may have a substituent, and a plurality of Rs's were present and the groups were different from each other, the electric field strength dependence of the response speed was more excellent.

### [Test Y]

Subsequently, in addition to the specific compound or the comparative compound, a photoelectric conversion element was produced using a coloring agent other than the specific compound, and the quantum efficiency (sensitivity) at a wavelength of 460 nm, the response speed, and the electric field strength dependence of the response speed of the photoelectric conversion element were evaluated by the following methods.

### <Production of Photoelectric Conversion Element>

A photoelectric conversion film 12 was formed by co-vapor depositing any specific compound selected from the compounds (B-1) to (B-23) or each comparative compound, the n-type organic semiconductor (fullerene (C₆₀)), the p-type organic semiconductor (compound (D-1)), and any coloring agent selected from (R-1)) to (R-11) in a ratio of specific compound:coloring agent:p-type organic semiconductor:n-type organic semiconductor=1:1:2:2 in terms of a single layer by a vacuum vapor deposition method, and a photoelectric conversion element of each of Examples and each of Comparative Examples was produced in the same manner for the other procedures as in Test X.

As a result, even in a case where the coloring agent other than the specific compound was used in combination, the same results as the results of the quantum efficiency (sensitivity), the response speed, and the electric field strength dependence of the response speed shown in Table 1 were obtained.

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1), 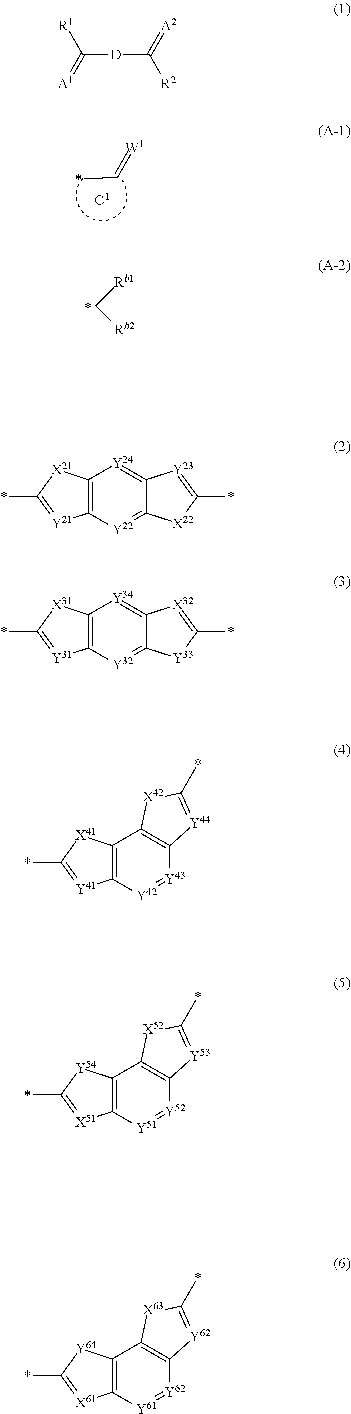
in Formula (1),
D represents a group represented by any of Formula (2) to Formula (6),
in Formula (2) to Formula (6), * represents a bonding position,
R¹ and R² each independently represent a hydrogen atom or a substituent, and
A¹ and A² each independently represent a group represented by Formula (A-1) or Formula (A-2),
in Formula (A-1) or Formula (A-2),
* represents a bonding position,
in Formula (2) to Formula (6),
X²¹ represents an oxygen atom, NR^{X1}, or CR^{X2}R^{X3},
X²², X³¹, X³², X⁴¹, X⁴², X⁵¹, X⁵², X⁶¹, and X⁶² each independently represent a sulfur atom, an oxygen atom, NR^{X1}, or CR^{X2}R^{X3},
R^{X1} to R^{X3} each independently represent a hydrogen atom, an aliphatic hydrocarbon group which may have a substituent, or an aromatic ring group which may have a substituent, and
Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ each independently represent —CR^{Y1}═ or a nitrogen atom, and R^{Y1} represents a hydrogen atom or a substituent,
in Formula (A-1),
C¹ represents a ring which contains two or more carbon atoms and may have a substituent,
W¹ represents a sulfur atom, an oxygen atom, =NR^{W2}, or =CR^{W3}R^{W4},
R^{W2} represents a hydrogen atom or a substituent,
R^{W3} and R^{W4} each independently represent a cyano group, —SO₂R^{W5}, —COOR^{W6}, or —COR^{W7}, and
R^{W5} to R^{W7} each independently represent an aliphatic hydrocarbon group which may have a substituent, an aromatic ring group which may have a substituent, or an aliphatic heterocyclic group which may have a substituent,
in Formula (A-2),
R^{b1} and R^{b2} each independently represent a cyano group, —COOR^{b3}, or —COR^{b4},
R^{b3} and R^{b4} each independently represent an aliphatic hydrocarbon group which may have a substituent, an aromatic ring group which may have a substituent, or an aliphatic heterocyclic group which may have a substituent, and
* represents a bonding position.

2. The photoelectric conversion element according to claim 1,
wherein at least one of Y²¹, . . . , or Y²⁴ in Formula (2) represents —CRs=, at least one of Y³¹, or Y³⁴ in Formula (3) represents —CRs=, at least one of Y⁴¹, . . . , or Y⁴⁴ in Formula (4) represents —CRs=, at least one of Y⁵¹, . . . , or Y⁵⁴ in Formula (5) represents —CRs=, and at least one of Y⁶¹, . . . , or Y⁶⁴ in Formula (6) represents —CRs=, and Rs's each independently represent a group selected from a substituent group S,
the substituent group S: an alkyl group which may have a substituent, an alkenyl group which may have a substituent, an alkynyl group which may have a substituent, an aryl group which may have a substituent, a heteroaryl group which may have a substituent, an alkoxy group which may have a substituent, an aryloxy group which may have a substituent, an amino group which may have a substituent, a cyano group, and a halogen atom,
in a case where a plurality of Rs's are present, Rs's may be the same or different from each other.

3. The photoelectric conversion element according to claim 2,
wherein D represents a group represented by any of Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), or Formula (6-12), 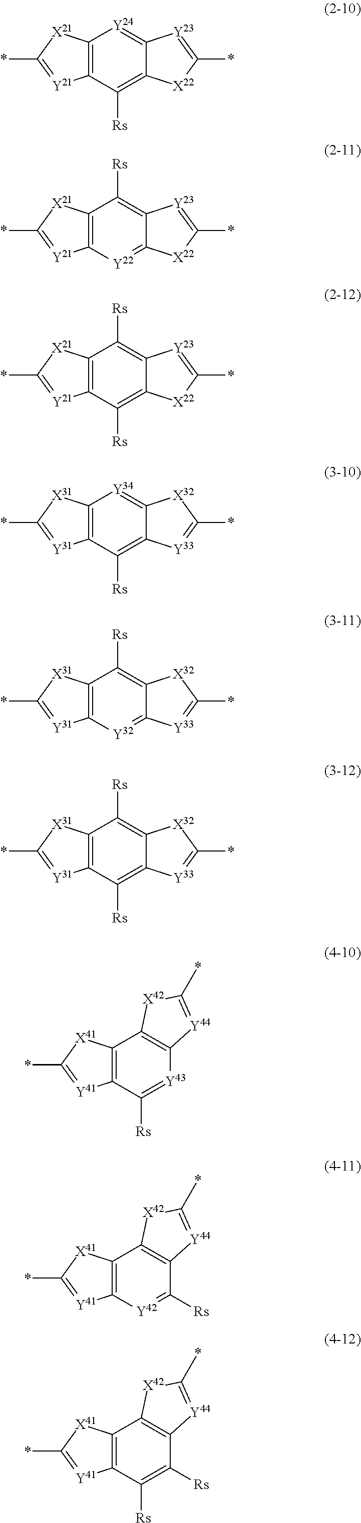 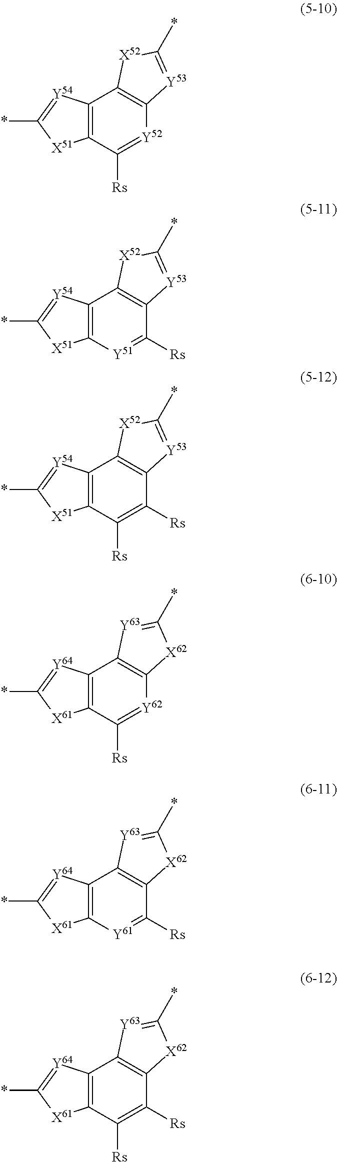
in Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), and Formula (6-12), * represents a bonding position,
X²¹ represents an oxygen atom, NR^{X1}, or CR^{X2}R^{X3},
X²², X³¹, X³², X⁴¹, X⁴², X⁵¹, X⁵², X⁶¹, and X⁶² each independently represent a sulfur atom, an oxygen atom, NR^{X1}, or CR^{X2}R^{X3},
R^{X1} to R^{X3} each independently represent a hydrogen atom, an aliphatic hydrocarbon group which may have a substituent, or an aromatic ring group which may have a substituent,
Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³ and Y⁶⁴ each independently represent —CR^{Y1}═ or a nitrogen atom, R^{Y1} represents a hydrogen atom or a substituent, and
Rs's each independently represent a group selected from a substituent group S,
the substituent group S: an alkyl group which may have a substituent, an alkenyl group which may have a substituent, an alkynyl group which may have a substituent, an aryl group which may have a substituent, a heteroaryl group which may have a substituent, an alkoxy group which may have a substituent, an aryloxy group which may have a substituent, an amino group which may have a substituent, a cyano group, and a halogen atom,
in Formula (2-12), Formula (3-12), Formula (4-12), Formula (5-12), and Formula (6-12), two Rs's may be the same group or different groups.

4. The photoelectric conversion element according to claim 1,
wherein Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ each independently represent —CR^{Y1}=.

5. The photoelectric conversion element according to claim 1,
wherein A¹ and A² are each independently the group represented by Formula (A-1).

6. The photoelectric conversion element according to claim 3,
wherein Rs represents a group selected from the group consisting of the alkyl group which may have a substituent, the aryl group which may have a substituent, the heteroaryl group which may have a substituent, and the halogen atom, and
in Formula (2-12), Formula (3-12), Formula (4-12), Formula (5-12), and Formula (6-12), two Rs's represent groups different from each other.

7. The photoelectric conversion element according to claim 6,
wherein Rs represents a group selected from the group consisting of the alkyl group which may have a substituent and the aryl group which may have a substituent, and
in Formula (2-12), Formula (3-12), Formula (4-12), Formula (5-12), and Formula (6-12), two Rs's represent groups different from each other.

8. The photoelectric conversion element according to claim 1,
wherein the photoelectric conversion film further contains an n-type organic semiconductor, and
the photoelectric conversion film has a bulk heterojunction structure formed in a state in which the compound represented by Formula (1) and the n-type organic semiconductor are mixed with each other.

9. The photoelectric conversion element according to claim 8,
wherein the n-type organic semiconductor includes fullerenes selected from the group consisting of a fullerene and derivatives of the fullerene.

10. The photoelectric conversion element according to claim 1,
wherein the photoelectric conversion film further contains a p-type organic semiconductor.

11. The photoelectric conversion element according to claim 1, further comprising:
one or more interlayers between the conductive film and the transparent conductive film, in addition to the photoelectric conversion film.

12. An imaging element comprising:
the photoelectric conversion element according to claim 1.

13. An optical sensor comprising:
the photoelectric conversion element according to claim 1.

14. A compound represented by Formula (1), 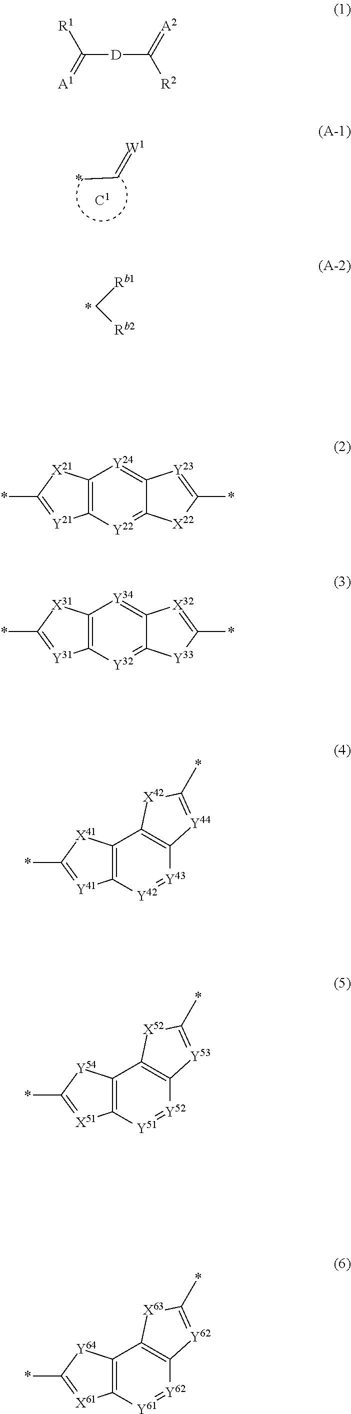
in Formula (1),
D represents a group represented by any of Formula (2) to Formula (6),
in Formula (2) to Formula (6), * represents a bonding position,
R¹ and R² each independently represent a hydrogen atom or a substituent,
A¹ and A² each independently represent a group represented by Formula (A-1) or Formula (A-2),
in Formula (A-1) or Formula (A-2),
* represents a bonding position,
in Formula (2) to Formula (6),
X²¹ represents an oxygen atom, NR^{X1}, or CR^{X2}R^{X3},
X²², X³¹, X³², X⁴¹, X⁴², X⁵¹, X⁵², X⁶¹, and X⁶² each independently represent a sulfur atom, an oxygen atom, NR^{X1}, or CR^{X2}R^{X3},
R^{X1} to R^{X3} each independently represent a hydrogen atom, an aliphatic hydrocarbon group which may have a substituent, or an aromatic ring group which may have a substituent, and
Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³ and Y⁶⁴ each independently represent —CR^{Y1}═ or a nitrogen atom, and R^{Y1} represents a hydrogen atom or a substituent,
in Formula (A-1),
C¹ represents a ring which contains two or more carbon atoms and may have a substituent,
W¹ represents a sulfur atom, an oxygen atom, =NR^{W2}, or =CR^{W3}R^{W4},
R^{W2} represents a hydrogen atom or a substituent,
R^{W3} and R^{W4} each independently represent a cyano group, —SO₂R^{W5}, —COOR^{W6}, or —COR^{W7}, and
R^{W5} to R^{W7} each independently represent an aliphatic hydrocarbon group which may have a substituent, an aromatic ring group which may have a substituent, or an aliphatic heterocyclic group which may have a substituent,
in Formula (A-2),
R^{b1} and R^{b2} each independently represent a cyano group, —COOR^{b3}, or —COR^{b4},
R^{b3} and R^{b4} each independently represent an aliphatic hydrocarbon group which may have a substituent, an aromatic ring group which may have a substituent, or an aliphatic heterocyclic group which may have a substituent, and
* represents a bonding position.

15. The compound according to claim 14,
wherein at least one of Y²¹, . . . , or Y²⁴ in Formula (2) represents —CRs=, at least one of Y³¹, . . . , or Y³⁴ in Formula (3) represents —CRs=, at least one of Y⁴¹, . . . , or Y⁴⁴ in Formula (4) represents —CRs=, at least one of Y⁵¹, . . . , or Y⁵⁴ in Formula (5) represents —CRs=, and at least one of Y⁶¹, . . . , or Y⁴ in Formula (6) represents —CRs=, and Rs's each independently represent a group selected from a substituent group S,
the substituent group S: an alkyl group which may have a substituent, an alkenyl group which may have a substituent, an alkynyl group which may have a substituent, an aryl group which may have a substituent, a heteroaryl group which may have a substituent, an alkoxy group which may have a substituent, an aryloxy group which may have a substituent, an amino group which may have a substituent, a cyano group, and a halogen atom,
in a case where a plurality of Rs's are present, Rs's may be the same or different from each other.

16. The compound according to claim 15,
wherein D represents a group represented by any of Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), or Formula (6-12), 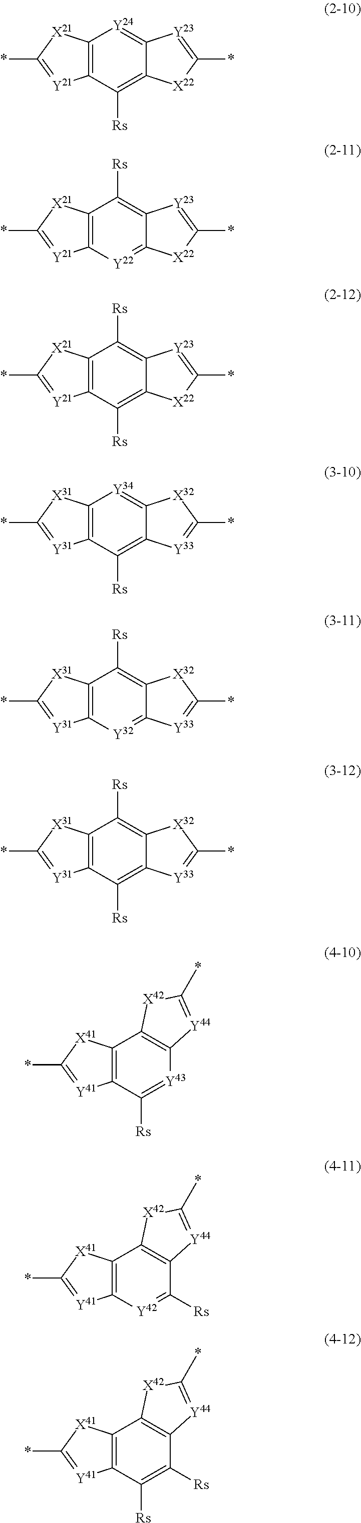 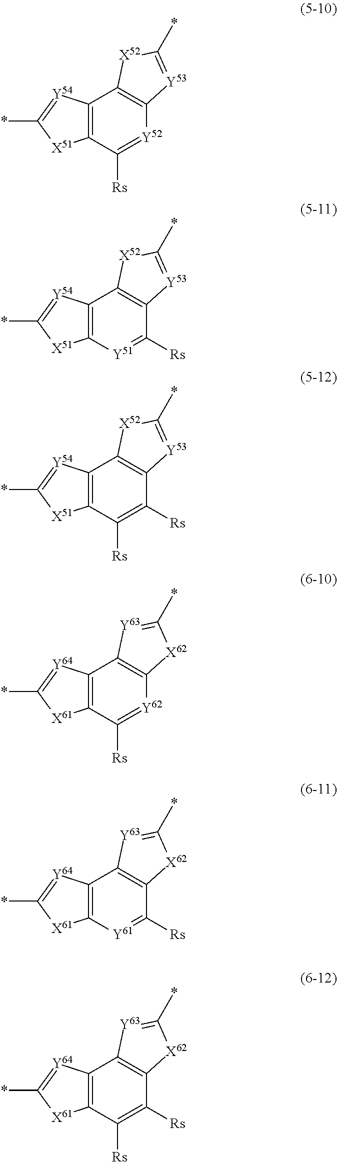
in Formula (2-10), Formula (2-11), Formula (2-12), Formula (3-10), Formula (3-11), Formula (3-12), Formula (4-10), Formula (4-11), Formula (4-12), Formula (5-10), Formula (5-11), Formula (5-12), Formula (6-10), Formula (6-11), and Formula (6-12), * represents a bonding position,
X²¹ represents an oxygen atom, NR^{X1}, or CR^{X2}R^{X3},
X²², X³¹, X³², X⁴¹, X⁴², X⁵¹, X⁵², X⁶¹, and X⁶² each independently represent a sulfur atom, an oxygen atom, NR^{X1}, or CR^{X2}R^{X3},
R^{X1} to R^{X3} each independently represent a hydrogen atom, an aliphatic hydrocarbon group which may have a substituent, or an aromatic ring group which may have a substituent,
Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵³, Y⁵⁴, Y⁶¹, Y⁶², Y⁶³, and Y⁶⁴ each independently represent —CR^{Y1}═ or a nitrogen atom, R^{Y1} represents a hydrogen atom or a substituent, and
Rs's each independently represent a group selected from a substituent group S,
the substituent group S: an alkyl group which may have a substituent, an alkenyl group which may have a substituent, an alkynyl group which may have a substituent, an aryl group which may have a substituent, a heteroaryl group which may have a substituent, an alkoxy group which may have a substituent, an aryloxy group which may have a substituent, an amino group which may have a substituent, a cyano group, and a halogen atom,
in Formula (2-12), Formula (3-12), Formula (4-12), Formula (5-12), and Formula (6-12), two Rs's may be the same group or different groups.

17. The compound according to claim 14,
wherein Y²¹, Y²², Y²³, Y²⁴, Y³¹, Y³², Y³³, Y³⁴, Y⁴¹, Y⁴², Y⁴³, Y⁴⁴, Y⁵¹, Y⁵², Y⁵³, Y⁵⁴, Y⁶¹, Y⁶², Y⁶³, and Y⁶⁴ each independently represent —CR^{Y1}=.

18. The compound according to claim 14,
wherein A¹ and A² are each independently the group represented by Formula (A-1).

19. The compound according to claim 16,
wherein Rs represents a group selected from the group consisting of the alkyl group which may have a substituent, the aryl group which may have a substituent, the heteroaryl group which may have a substituent, and the halogen atom, and
in Formula (2-12), Formula (3-12), Formula (4-12), Formula (5-12), and Formula (6-12), two Rs's represent groups different from each other.

20. The compound according to claim 19,
wherein Rs represents a group selected from the group consisting of the alkyl group which may have a substituent and the aryl group which may have a substituent, and
in Formula (2-12), Formula (3-12), Formula (4-12), Formula (5-12), and Formula (6-12), two Rs's represent groups different from each other.
